(19) United States
(12) Patent Application Publication
(10) Pub. No.: US 2025/0034183 A1
(43) Pub. Date: Jan. 30, 2025

(54) POLYMORPHIC FORMS AND METHODS OF PRODUCING POLYMORPHIC FORMS OF A COMPOUND

(71) Applicant: Viking Therapeutics, Inc., San Diego, CA (US)

(72) Inventors: Geoffrey E. Barker, Carlsbad, CA (US); Maureen Barnes, San Diego, CA (US)

(21) Appl. No.: 18/836,713

(22) PCT Filed: Feb. 13, 2023
(86) PCT No.: PCT/US2023/012898

§ 371 Date: Aug. 7, 2024

Related U.S. Application Data
(60) Provisional application No. 63/311,869, filed on Feb. 18, 2022.

(51) Int. Cl. C07F 9/38; A61K 31/662

(52) U.S. Cl. CPC C07F 9/3808; A61K 31/662; C07B 2200/13

(57) ABSTRACT

Disclosed herein are thyroid receptor agonist compounds, polymorphic forms, pharmaceutical compositions, the method of use, and preparation thereof. Some embodiments relate to crystalline forms of Compound 1 and salts thereof.

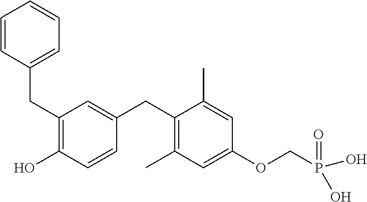 [SMILES: Cc1cc(OCP(=O)(O)O)cc(C)c1Cc1ccc(O)c(Cc2ccccc2)c1]

### BACKGROUND

### Field

The present application relates to the fields of chemistry and medicine. More particularly, the present application relates thyroid agonist compounds, polymorphic forms, pharmaceutical compositions thereof, and their use as therapeutic agents.

### Description of the Related Art

Thyroid hormones (TH) are synthesized in the thyroid in response to thyroid stimulating hormone (TSH), which is secreted by the pituitary gland in response to various stimulants (e.g., thyrotropin-releasing hormone (TRH) from the hypothalamus). Thyroid hormones are iodinated O-aryl tyrosine analogues excreted into the circulation primarily as 3,3′,5,5′-tetraiodothyronine (T4). T4 is rapidly deiodinated in local tissues by thyroxine 5′-deiodinase to 3,3′,5′-triiodothyronine (T3), which is the most potent TH. T3 is metabolized to inactive metabolites via a variety of pathways, including pathways involving deiodination, glucuronidation, sulfation, deamination, and decarboxylation. Most of the circulating T4 and T3 is eliminated through the liver.

The biological activity of THs is mediated largely through thyroid hormone receptors (TRs). TRs belong to the nuclear receptor superfamily, which, along with its common partner, the retinoid X receptor, form heterodimers that act as ligand-inducible transcription factors. Like other nuclear receptors, TRs have a ligand binding domain and a DNA binding domain and regulate gene expression through ligand-dependent interactions with DNA response elements (thyroid response elements, TREs). Currently, the literature shows that TRs are encoded by two distinct genes (TRα and TRβ), which produce several isoforms through alternative splicing (Williams, Mol. Cell. Biol. 20(22):8329-42 (2000); Nagaya et al., Biochem. Biophys. Res. Commun. 226(2):426-30 (1996)). The major isoforms that have so far been identified are TRα-1, TRα-2, TRβ1-1 and TRs-2. TRα-1 is ubiquitously expressed in the rat with highest expression in skeletal muscle and brown fat. TRβ-1 is also ubiquitously expressed with highest expression in the liver, brain and kidney. TRβ-2 is expressed in the anterior pituitary gland and specific regions of the hypothalamus as well as the developing brain and inner ear. In the rat and mouse liver, TRβ-1 is the predominant isoform (80%). The TR isoforms found in human and rat are highly homologous with respect to their amino acid sequences which suggest that each serves a specialized function.

TH's affect the growth, metabolism and the physiological function of nearly all organs. TH's lower serum cholesterol and triglycerides. However, side effects of TH action include cardiac arrhythmia, bone loss, nervousness, and anxiety.

TRβ agonists may be useful as therapeutics for conditions such as hepatitis, non-alcoholic fatty liver disease, non-alcoholic steatohepatitis (NASH), and a variety of fibrotic disease and disorders. Accordingly, a need exists to provide TRβ agonist compounds with suitable stability properties for therapeutic use.

### SUMMARY

In a first aspect of the present disclosure, provided herein is a crystalline form of Compound 1:

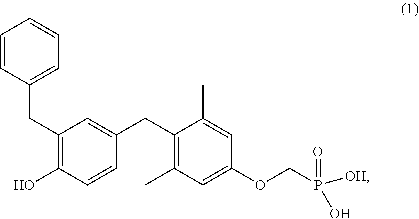 [SMILES: Cc1cc(OCP(=O)(O)O)cc(C)c1Cc1ccc(O)c(Cc2ccccc2)c1]

or a solvate thereof.

In some embodiments of the first aspect, the crystalline form may exhibit an X-ray powder diffraction pattern comprising at least one characteristic peak, wherein said characteristic peak is selected from the group consisting of approximately 5.1, 7.4, 10.1, 12.1, 16.4, 17.3, 18.5, 20.5, 21.4, 21.7, 22.1, 23.8, 24.1, 25.5, and 25.9 degrees 2θ. In some embodiments, the crystalline form may exhibit an X-ray powder diffraction pattern comprising at least three characteristic peaks, wherein said characteristic peaks are selected from the group consisting of 5.1, 7.4, 10.1, 12.1, 16.4, 17.3, 18.5, 20.5, 21.4, 21.7, 22.1, 23.8, 24.1, 25.5, and 25.9 degrees 2θ. In some embodiments, the crystalline form may have a melting point of about 161° C.

In some embodiments, provided herein is a crystalline form of Compound 1, wherein the crystalline form may exhibit an X-ray powder diffraction pattern comprising at least one characteristic peak, wherein said characteristic peak is selected from the group consisting of approximately 7.5, 8.1, 10.0, 15.4, 16.7, 18.3, 18.8, 20.2, 21.0, 22.3, 24.1, 25.1, and 26.4 degrees 2θ. In some embodiments, the crystalline form may exhibit an X-ray powder diffraction pattern comprising at least three characteristic peaks, wherein said characteristic peaks are selected from the group consisting of 7.5, 8.1, 10.0, 15.4, 16.7, 18.3, 18.8, 20.2, 21.0, 22.3, 24.1, 25.1, and 26.4 degrees 2θ.

In some embodiments, provided herein is a crystalline form of Compound 1, wherein the crystalline form may exhibit an X-ray powder diffraction pattern comprising at least one characteristic peak, wherein said characteristic peak is selected from the group consisting of approximately 4.0, 8.0, 11.0, 12.0, 14.4, 14.6, 17.1, 18.5, 19.2, 21.1, 21.7, 24.9, and 25.7 degrees 2θ. In some embodiments, the crystalline form may exhibit an X-ray powder diffraction pattern comprising at least three characteristic peaks, wherein said characteristic peaks are selected from the group consisting of 4.0, 8.0, 11.0, 12.0, 14.4, 14.6, 17.1, 18.5, 19.2, 21.1, 21.7, 24.9, and 25.7 degrees 2θ. In some embodiments, the crystalline form may have a DSC endotherm at about 70° C. or 158° C.

In some embodiments, provided herein is a crystalline of Compound 1, wherein the crystalline form may exhibit an X-ray powder diffraction pattern comprising at least one characteristic peak, wherein said characteristic peak is selected from the group consisting of approximately 76.1, 7.6, 11.3, 12.2, 16.5, 17.0, 17.3, 18.4, 18.6, 19.5, 19.7, 20.7, 21.2, 22.5, 26.0, and 26.3 degrees 2θ. In some embodiments, the crystalline form may exhibit an X-ray powder diffraction pattern comprising at least three characteristic peaks, wherein said characteristic peaks are selected from the group consisting of 6.1, 7.6, 11.3, 12.2, 16.5, 17.0, 17.3, 18.4, 18.6, 19.5, 19.7, 20.7, 21.2, 22.5, 26.0, and 26.3 degrees 2θ. In some embodiments, the crystalline form may have a DSC endotherm at about 166° C.

In some embodiments, provided herein is a crystalline form of Compound 1, wherein the crystalline form may exhibit an X-ray powder diffraction pattern comprising at least one characteristic peak, wherein said characteristic peak is selected from the group consisting of approximately 3.3, 5.0, 7.5, 14.9, 16.3, 17.2, 19.4, 21.7, 24.7, 25.5, and 26.6 degrees 2θ. In some embodiments, the crystalline form may exhibit an X-ray powder diffraction pattern comprising at least three characteristic peaks, wherein said characteristic peaks are selected from the group consisting of 3.3, 5.0, 7.5, 14.9, 16.3, 17.2, 19.4, 21.7, 24.7, 25.5, and 26.6 degrees 2θ. In some embodiments, the crystalline form may have a DSC endotherm at about 96° C. or 166° C.

In some embodiments, provided herein is a crystalline of Compound 1, wherein the crystalline form may exhibit an X-ray powder diffraction pattern comprising at least one characteristic peak, wherein said characteristic peak is selected from the group consisting of approximately 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ. In some embodiments, the crystalline form may exhibit an X-ray powder diffraction pattern comprising at least three characteristic peaks, wherein said characteristic peaks are selected from the group consisting of 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ. In some embodiments, the crystalline form may have a DSC endotherm at about 96° C.

In some embodiments of the first aspect provided herein, the crystalline form may be unsolvated. In other embodiments, the crystalline form may be solvated.

In a second aspect of the present disclosure, provided herein is a crystalline form of Compound 1-A:

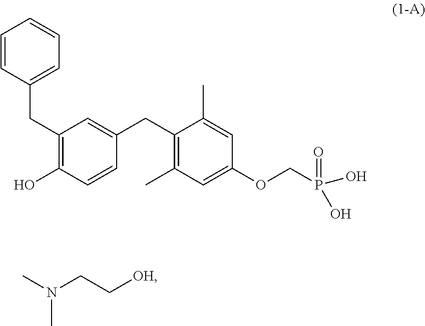

or a solvate thereof.

In some embodiments, the crystalline form of Compound 1-A may exhibit an X-ray powder diffraction pattern comprising at least one characteristic peak, wherein said characteristic peak is selected from the group consisting of approximately 6.0, 13.3, 14.4, 14.8, 16.1, 17.8, 17.9, 18.7, 20.5, 20.8, 21.7, 22.9, 23.8, 24.9, 25.1, and 26.6 degrees 2θ. In some embodiments, the crystalline form may exhibit an X-ray powder diffraction pattern comprising at least three characteristic peaks, wherein said characteristic peaks are selected from the group consisting of 6.0, 13.3, 14.4, 14.8, 16.1, 17.8, 17.9, 18.7, 20.5, 20.8, 21.7, 22.9, 23.8, 24.9, 25.1, and 26.6 degrees 2θ. In some embodiments, the crystalline form may have a melting point of about 148° C. In some embodiments the crystalline form may have a melting point at about 152° C. In some embodiments, the crystalline form may have a melting point at about 156° C. In some embodiments, the crystalline form may have a melting point at from about 146° C. to about 158° C. In some embodiments, crystalline form may have a melting point at from about 148° C. to about 156° C. In some embodiments, crystalline form may have a DSC melting point at from about 146° C. to about 150° C. In some embodiments, crystalline form may have a melting point at from about 152° C. to about 156° C.

In some embodiments, the crystalline form of Compound 1-A may exhibit an X-ray powder diffraction pattern comprising at least one characteristic peak, wherein said characteristic peak is selected from the group consisting of approximately 6.1, 12.2, 13.3, 13.6, 16.5, 17.3, 18.3, 19.9, 20.2, 20.5, 21.4, 21.9, 22.5, 22.8, 23.0, and 25.6 degrees 2θ. In some embodiments, the crystalline form may exhibit an X-ray powder diffraction pattern comprising at least three characteristic peaks, wherein said characteristic peaks are selected from the group consisting of 6.1, 12.2, 13.3, 13.6, 16.5, 17.3, 18.3, 19.9, 20.2, 20.5, 21.4, 21.9, 22.5, 22.8, 23.0, and 25.6 degrees 2θ. In some embodiments, wherein the crystalline form may have a melting point of about 139° C.

In some embodiments, the crystalline form of Compound 1-A may exhibits an X-ray powder diffraction pattern comprising at least one characteristic peak, wherein said characteristic peak is selected from the group consisting of approximately 5.7, 6.2, 15.5, 16.4, 17.0, 18.5, 20.6, 21.2, 22.0, 23.3, and 26.1 degrees 2θ. In some embodiments, the crystalline form may exhibit an X-ray powder diffraction pattern comprising at least three characteristic peaks, wherein said characteristic peaks are selected from the group consisting of 5.7, 6.2, 15.5, 16.4, 17.0, 18.5, 20.6, 21.2, 22.0, 23.3, and 26.1 degrees 2θ. In some embodiments, wherein the crystalline form may have a melting point of about 117° C.

In some embodiments, the crystalline form of Compound 1-A may exhibit an X-ray powder diffraction pattern comprising at least one characteristic peak, wherein said characteristic peak is selected from the group consisting of approximately 6.2, 12.9, 14.0, 14.5, 16.5, 17.6, 18.0, 18.6, 20.3, 21.2, 22.7, 23.3, 24.0, and 26.1 degrees 2θ. In some embodiments, the crystalline form may exhibit an X-ray powder diffraction pattern comprising at least three characteristic peaks, wherein said characteristic peaks are selected from the group consisting of 6.2, 12.9, 14.0, 14.5, 16.5, 17.6, 18.0, 18.6, 20.3, 21.2, 22.7, 23.3, 24.0, and 26.1 degrees 2θ. In some embodiments, the crystalline form may a melting point of about 114° C.

In some embodiments of the first aspect provided herein, the crystalline form of Compound 1-A may be unsolvated. In other embodiments, the crystalline form of Compound 1-A may be solvated. In some specific embodiments, the crystalline form of Compound 1-A may be a hydrate. In some specific embodiments, the crystalline form of Compound 1-A may be a monohydrate.

Also provided herein is a pharmaceutical composition comprising a therapeutically effective amount of one or more crystalline forms described herein and one or more pharmaceutically acceptable excipients.

Also provided herein is a method of treating a disease or condition in a subject, the method comprising administering to the subject a therapeutically effective amount of a crystalline form described herein, wherein said disease or disorder is selected from the group consisting of steatosis, non-alcoholic fatty liver disease (NAFLD), non-alcoholic steatohepatitis (NASH), liver fibrosis, renal fibrosis, biliary fibrosis, pancreatic fibrosis, chronic kidney disease, diabetic kidney disease, primary sclerosing cholangitis, primary biliary cirrhosis, or idiopathic fibrosis.

In some embodiments, wherein the method may result in the prevention, treatment, or amelioration, of a fibrosis, fibrotic condition, or fibrotic symptoms. In some embodiments, the method may result in the reduction in the amount of extracellular matrix proteins present in one or more tissues of said subject. In some embodiments, the method may result in the reduction in the amount of collagen present in one or more tissues of said subject. In some embodiments, the administration of the crystal forms described herein may result in the reduction in the amount of Type I, Type Ia, or Type III collagen present in one or more tissues of said subject.

In some embodiments, described herein is method of treating a disease or disorder in a subject, the method comprising administering to the subject a therapeutically effective amount of a solution prepared by dissolving a crystalline form described herein in a pharmaceutically acceptable solvent, wherein said disease or disorder is selected from the group consisting of steatosis, non-alcoholic fatty liver disease (NAFLD), non-alcoholic steatohepatitis (NASH), liver fibrosis, renal fibrosis, biliary fibrosis, pancreatic fibrosis, chronic kidney disease, diabetic kidney disease, primary sclerosing cholangitis, primary biliary cirrhosis, or idiopathic fibrosis.

In another aspect of the present disclosure, described herein is compound, having the structure

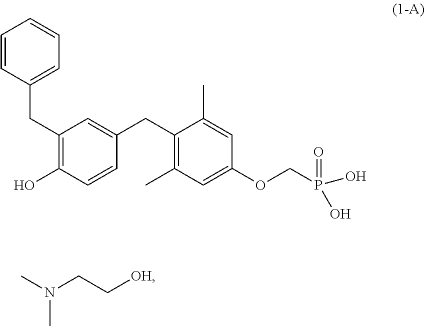

or a solvate thereof.

### BRIEF DESCRIPTION OF THE DRAWINGS

FIG. 1 is an X-ray powder diffraction pattern of crystalline Form A of Compound 1.

FIG. 2 shows a differential scanning calorimetry and thermogravimetric analysis overlay for crystalline Form A of compound of Compound 1.

FIG. 3 shows moisture sorption/desorption curves or crystalline Form A of compound of Compound 1.

FIG. 4 is an X-ray powder diffraction pattern of crystalline Form B of Compound 1.

FIG. 5 is an X-ray powder diffraction pattern of crystalline Form C of Compound 1.

FIG. 6 shows a differential scanning calorimetry and thermogravimetric analysis overlay for crystalline Form C of compound of Compound 1.

FIG. 7 is an X-ray powder diffraction pattern of crystalline Form D of Compound 1.

FIG. 8 shows a differential scanning calorimetry and thermogravimetric analysis overlay for crystalline Form D of compound of Compound 1.

FIG. 9 is an X-ray powder diffraction pattern of crystalline Form E of Compound 1.

FIG. 10 shows a differential scanning calorimetry and thermogravimetric analysis overlay for crystalline Form E of compound of Compound 1.

FIG. 11 is an X-ray powder diffraction pattern of crystalline Form F of Compound 1.

FIG. 12 shows a differential scanning calorimetry and thermogravimetric analysis overlay for crystalline Form F of compound of Compound 1.

FIG. 13 is an X-ray powder diffraction pattern of crystalline Form 1 of Compound 1-A.

FIG. 14 is an X-ray powder diffraction pattern of crystalline Form 2 of Compound 1-A.

FIG. 15 is an X-ray powder diffraction pattern of crystalline Form 3 of Compound 1-A.

FIG. 16 shows a differential scanning calorimetry and thermogravimetric analysis overlay for crystalline Form 3 of compound of Compound 1-A.

FIG. 17 is an X-ray powder diffraction pattern of crystalline Form 4 of Compound 1-A.

FIG. 18 shows a differential scanning calorimetry and thermogravimetric analysis overlay for crystalline Form 4 of compound of Compound 1-A.

### DETAILED DESCRIPTION

Disclosed herein are crystalline forms of Compound 1, or solvates thereof, and methods of crystallizing Compound. Compound 1 is shown below:

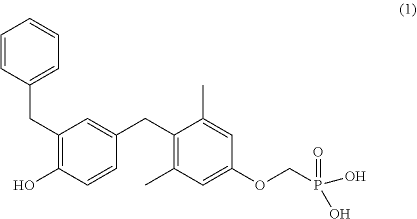 [SMILES: Cc1cc(OCP(=O)(O)O)cc(C)c1Cc1ccc(O)c(Cc2ccccc2)c1]

Crystalline forms of Compound 1 include Forms A, B, C, D, E, and F (described herein).

Disclosed herein is the (2-dimethylamino)ethanol (“deanol”) salt of Compound 1, referred to herein as Compound 1-A.

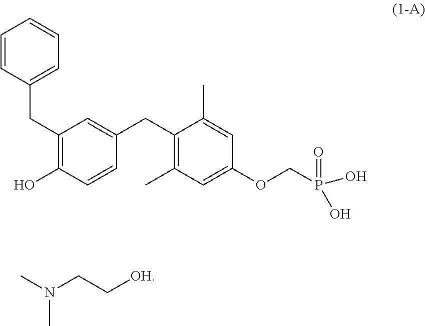

Also disclosed herein are crystalline forms of the deanol salt of Compound 1.

Crystalline forms of Compound 1-A include Forms 1, 2, 3, and 4 (described herein).

The present application relates to the first crystalline forms of Compound 1, as well as methods of crystallizing the various crystalline forms of Compound 1.

The present application relates to the first crystalline forms of Compound 1-A, as well as methods of crystallizing the various crystalline forms of Compound 1.-A

The crystalline forms provided herein advantageously exhibit improved solubility, bioavailability, stability, processability and ease of manufacture. As a result, Compound 1, particularly crystalline Form A, and Compound 1-A, particularly crystalline Form 3, provide improved long-term stability. Accordingly, the crystalline forms provide significant clinical improvements as TRβ agonists.

The present application also relates to the method using the crystalline forms described herein, for treating diseases and disorders by administering to a patient a therapeutically effective amount of a composition comprising one or more crystalline forms of Compound 1 and/or Compound 1-A, and one or more pharmaceutically acceptable excipients.

### Crystalline Forms of Compound 1

Disclosed herein are crystalline forms of Compound 1 and in particular crystalline Form A, crystalline Form B, crystalline Form C, crystalline Form D, crystalline Form E, and crystalline Form F. (described below). Unless otherwise stated, the X-ray powder diffraction data provided herein was determined using a Cu Kα radiation source.

### Crystalline Form A

Some embodiments include an unsolvated crystalline form of Compound 1, referred to herein as crystalline Form A. The precise conditions for forming crystalline Form A may be empirically determined and it is only possible to give a number of methods which have been found to be suitable in practice.

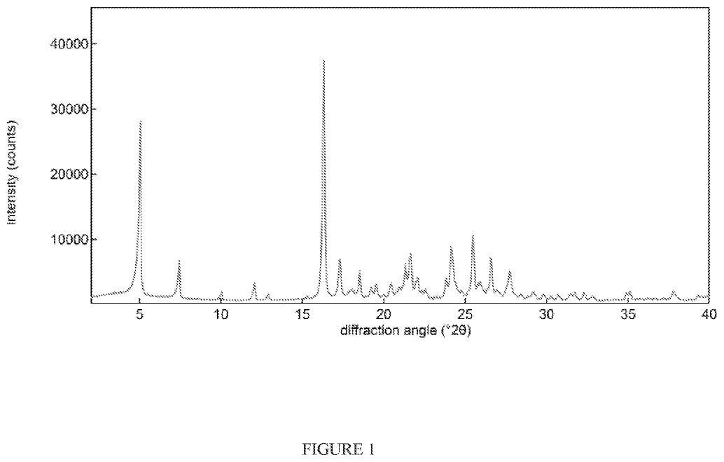

Crystalline Form A was characterized using various techniques which are described in further detail in the experimental methods section. FIG. 1 shows the crystalline structure of Form A as determined by X-ray powder diffraction (XRPD). Crystalline Form A, which may be obtained by the methods disclosed herein, exhibits prominent peaks at approximately 5.1, 7.4, 10.1, 12.1, 16.4, 17.3, 18.5, 20.5, 21.4, 21.7, 22.1, 23.8, 24.1, 25.5, and 25.9 degrees 2θ. Thus, in some embodiments, a crystalline form of Compound 1 has at least one characteristic peak (e.g., one, two, three, four, five, six, seven, eight, nine, ten, eleven, twelve, thirteen, fourteen, or fifteen characteristic peaks) selected from approximately 5.1, 7.4, 10.1, 12.1, 16.4, 17.3, 18.5, 20.5, 21.4, 21.7, 22.1, 23.8, 24.1, 25.5, and 25.9 degrees 2θ. In some embodiments, a crystalline form of Compound 1 has at least three characteristic peaks selected from approximately 5.1, 7.4, 10.1, 12.1, 16.4, 17.3, 18.5, 20.5, 21.4, 21.7, 22.1, 23.8, 24.1, 25.5, and 25.9 degrees 2θ.

As is well understood in the art, because of the experimental variability when X-ray diffraction patterns are measured on different instruments, the peak positions are assumed to be equal if the two theta (20) values agree to within a certain degree of variability. For example, the United States Pharmacopeia states that if the angular setting of the 10 strongest diffraction peaks agree to within ±0.2 degrees with that of a reference material, and the relative intensities of the peaks do not vary by more than 20%, the identity is confirmed. Accordingly, in some embodiments, peak positions recited herein include variability within ±0.5 degrees 2θ. In other embodiments, peak positions recited herein include variability within ±0.2 degrees 2θ. As disclosed herein, the term “approximately” when referring to values of 20 is defined as ±0.5 degrees 2θ.

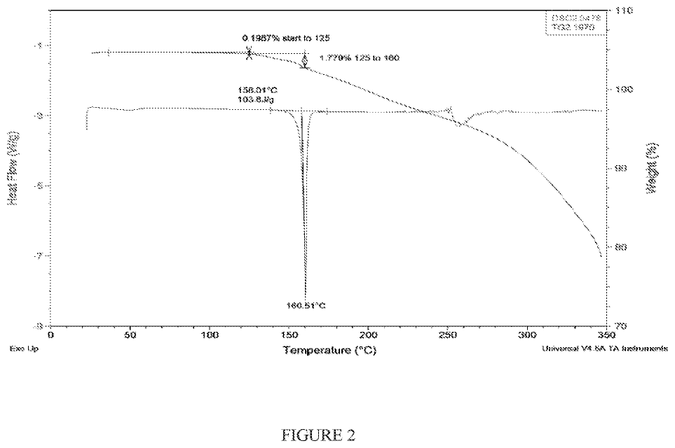

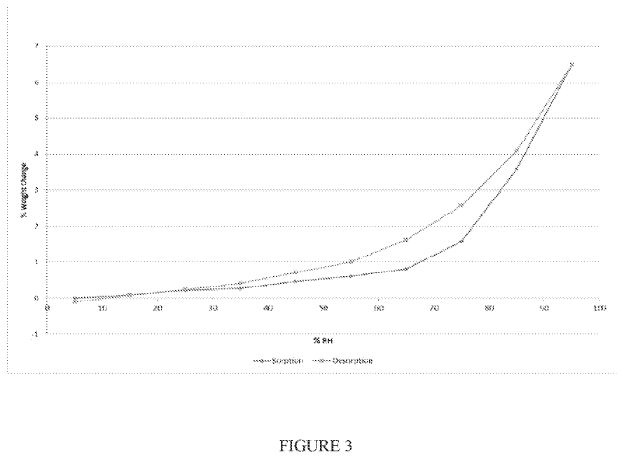

FIG. 2 shows results obtained by differential scanning calorimetry (DSC) and thermogravimetric analysis for crystalline Form A. The DSC results indicate a peak at a temperature of about 161° C. for Crystalline Form A, which indicates the melting point for the crystal. Accordingly, in some embodiments, Crystalline Form A exhibits a melting point from about 158° C. to 164° C., from about 158° C. to about 166° C., or at about 161° C. Crystalline Form A was analysed by thermogravimetric gravimetric analysis (TG), and in one instance exhibited a 1.78% weight loss when carried out from 25° C. to 160° C. Meanwhile, FIG. 3 shows dynamic vapor sorption (DVS) results for Crystalline Form A, and shows moderate water uptake.

Crystalline Form A can therefore be characterized as moderately hygroscopic and stable over a wide range of humidity. Crystalline Form A also shows good crystallinity and melting point is relatively high (approx. 161° C.) and crystal Form A does not show any evidence of hydrate formation.

### Crystalline Form B

Some embodiments include a solvated crystalline form of Compound 1, referred to herein as crystalline Form B. The precise conditions for forming crystalline Form B may be empirically determined and it is only possible to give a number of methods which have been found to be suitable in practice.

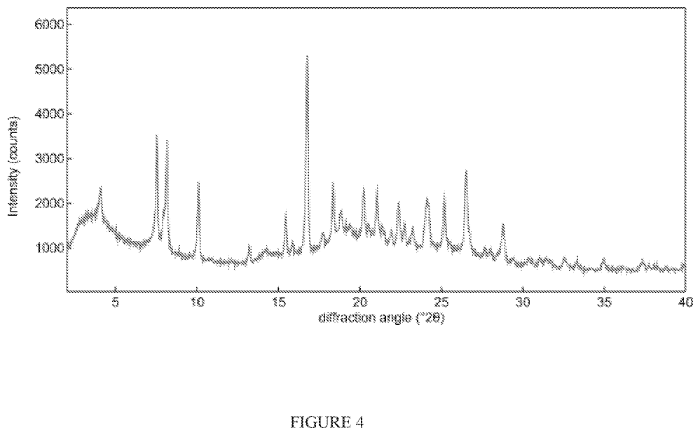

Crystalline Form B was characterized using various techniques which are described in further detail in the experimental methods section. FIG. 4 shows the crystalline structure of Form B as determined by X-ray powder diffraction (XRPD). Crystalline Form B, which may be obtained by the methods disclosed herein, exhibits prominent peaks at approximately 7.5, 8.1, 10.0, 15.4, 16.7, 18.3, 18.8, 20.2, 21.0, 22.3, 24.1, 25.1, and 26.4 degrees 2θ. Thus, in some embodiments, a crystalline form of Compound 1 has at least one characteristic peak (e.g., one, two, three, four, five, six, seven, eight, nine, ten, eleven, twelve, or thirteen characteristic peaks) selected from approximately 7.5, 8.1, 10.0, 15.4, 16.7, 18.3, 18.8, 20.2, 21.0, 22.3, 24.1, 25.1, and 26.4 degrees 2θ. In some embodiments, a crystalline form of Compound 1 has at least three characteristic peaks selected from approximately 7.5, 8.1, 10.0, 15.4, 16.7, 18.3, 18.8, 20.2, 21.0, 22.3, 24.1, 25.1, and 26.4 degrees 2θ.

Crystalline Form B shows evidence of solvate formation and is likely a solvate of isopropyl alcohol.

### Crystalline Form C

Some embodiments include a crystalline form of Compound 1, referred to herein as crystalline Form C. The precise conditions for forming crystalline Form C may be empirically determined and it is only possible to give a number of methods which have been found to be suitable in practice.

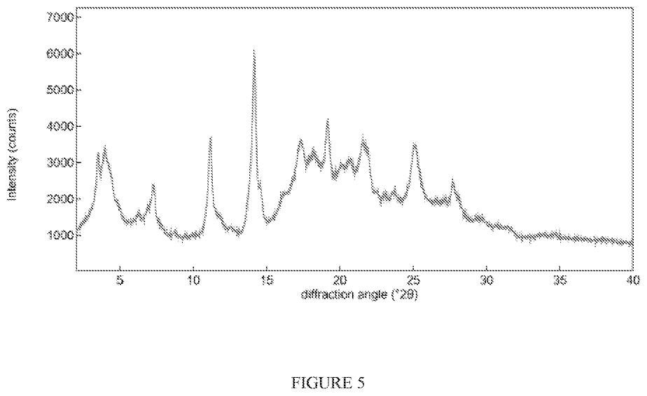

Crystalline Form C was characterized using various techniques which are described in further detail in the experimental methods section. FIG. 5 shows the crystalline structure of Form C as determined by X-ray powder diffraction (XRPD). Crystalline Form C, which may be obtained by the methods disclosed herein, exhibits prominent peaks at approximately 4.0, 8.0, 11.0, 12.0, 14.4, 14.6, 17.1, 18.5, 19.2, 21.1, 21.7, 24.9, and 25.7 degrees 2θ. Thus, in some embodiments, a crystalline form of Compound 1 has at least one characteristic peak (e.g., one, two, three, four, five, six, seven, eight, nine, ten, eleven, twelve, or thirteen characteristic peaks) selected from approximately 4.0, 8.0, 11.0, 12.0, 14.4, 14.6, 17.1, 18.5, 19.2, 21.1, 21.7, 24.9, and 25.7 degrees 2θ. In some embodiments, a crystalline form of Compound 1 has at least three characteristic peaks selected from approximately 4.0, 8.0, 11.0, 12.0, 14.4, 14.6, 17.1, 18.5, 19.2, 21.1, 21.7, 24.9, and 25.7 degrees 2θ.

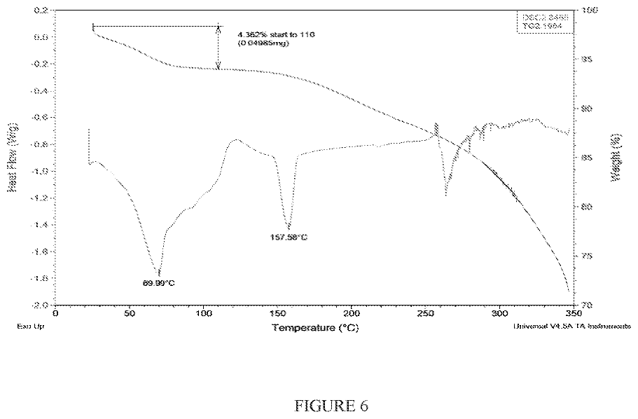

FIG. 6 shows results obtained by differential scanning calorimetry (DSC) and thermogravimetric analysis for crystalline Form C. The DSC results indicates endotherms at 70° C. and 158° C. for crystalline Form C. Crystalline Form C was analysed by thermogravimetric gravimetric analysis (TG), and in one instance exhibited a 4.4% weight loss when carried out from 25° C. to 110° C. Crystalline Form C becomes non-crystalline when heated to 115° C.

### Crystalline Form D

Some embodiments include a crystalline form of Compound 1, referred to herein as crystalline Form D. The precise conditions for forming crystalline Form D may be empirically determined and it is only possible to give a number of methods which have been found to be suitable in practice.

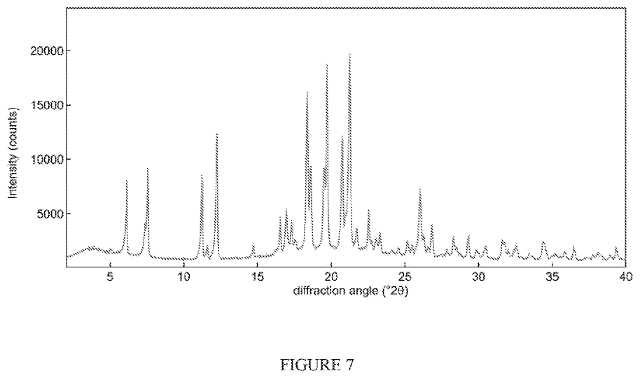

Crystalline Form D was characterized using various techniques which are described in further detail in the experimental methods section. FIG. 7 shows the crystalline structure of Form D as determined by X-ray powder diffraction (XRPD). Crystalline Form D, which may be obtained by the methods disclosed herein, exhibits prominent peaks at approximately 6.1, 7.6, 11.3, 12.2, 16.5, 17.0, 17.3, 18.4, 18.6, 19.5, 19.7, 20.7, 21.2, 22.5, 26.0, and 26.3 degrees 2θ. Thus, in some embodiments, a crystalline form of Compound 1 has at least one characteristic peak (e.g., one, two, three, four, five, six, seven, eight, nine, ten, eleven, twelve, thirteen, fourteen, fifteen, or sixteen characteristic peaks) selected from approximately 6.1, 7.6, 11.3, 12.2, 16.5, 17.0, 17.3, 18.4, 18.6, 19.5, 19.7, 20.7, 21.2, 22.5, 26.0, and 26.3 degrees 2θ. In some embodiments, a crystalline form of Compound 1 has at least three characteristic peaks selected from approximately 6.1, 7.6, 11.3, 12.2, 16.5, 17.0, 17.3, 18.4, 18.6, 19.5, 19.7, 20.7, 21.2, 22.5, 26.0, and 26.3 degrees 2θ.

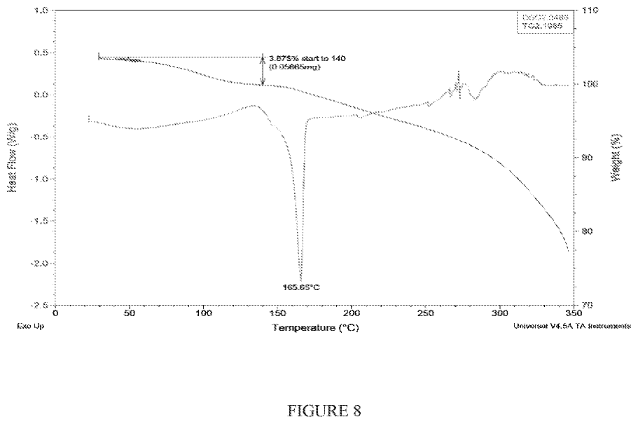

FIG. 8 shows results obtained by differential scanning calorimetry (DSC) and thermogravimetric analysis for crystalline Form D. The DSC results indicate an endotherm at 166° C. for crystalline Form D, which indicates the melting point for the crystal. Crystalline Form D was analysed by thermogravimetric gravimetric analysis (TG), and in one instance exhibited a 3.875% weight loss when carried out from 25° C. to 140° C., indicating a possible hydrate.

### Crystalline Form E

Some embodiments include an unsolvated crystalline form of Compound 1, referred to herein as crystalline Form E. The precise conditions for forming crystalline Form E may be empirically determined and it is only possible to give a number of methods which have been found to be suitable in practice.

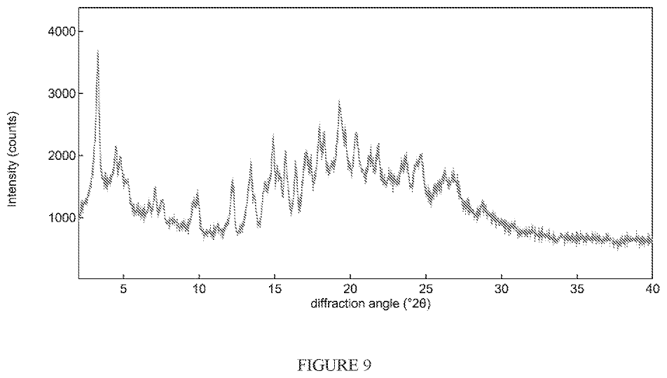

Crystalline Form E was characterized using various techniques which are described in further detail in the experimental methods section. FIG. 9 shows the crystalline structure of Form E as determined by X-ray powder diffraction (XRPD). Crystalline Form E, which may be obtained by the methods disclosed herein, exhibits prominent peaks at approximately 3.3, 5.0, 7.5, 14.9, 16.3, 17.2, 19.4, 21.7, 24.7, 25.5, and 26.6 degrees 2θ. Thus, in some embodiments, a crystalline form of Compound 1 has at least one characteristic peak (e.g., one, two, three, four, five, six, seven, eight, nine, ten, or eleven characteristic peaks) selected from approximately 3.3, 5.0, 7.5, 14.9, 16.3, 17.2, 19.4, 21.7, 24.7, 25.5, and 26.6 degrees 2θ. In some embodiments, a crystalline form of Compound 1 has at least three characteristic peaks selected from approximately 3.3, 5.0, 7.5, 14.9, 16.3, 17.2, 19.4, 21.7, 24.7, 25.5, and 26.6 degrees 2θ.

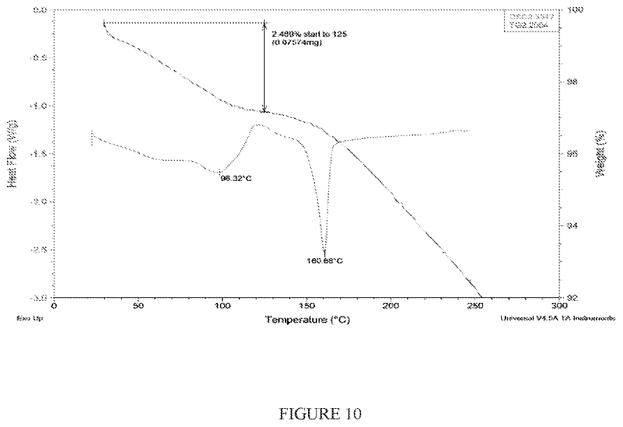

FIG. 10 shows results obtained by differential scanning calorimetry (DSC) and thermogravimetric analysis for crystalline Form E. The DSC results indicate endotherms at 96° C. and 166° C. for crystalline Form E. Crystalline Form E was analysed by thermogravimetric gravimetric analysis (TG), and in one instance exhibited a 2.5% weight loss when carried out from 25° C. to 125° C., however, the loss of mass is not attributed to solvent.

### Crystalline Form F

Some embodiments include a solvated crystalline form of Compound 1, referred to herein as crystalline Form F. The precise conditions for forming crystalline Form F may be empirically determined and it is only possible to give a number of methods which have been found to be suitable in practice.

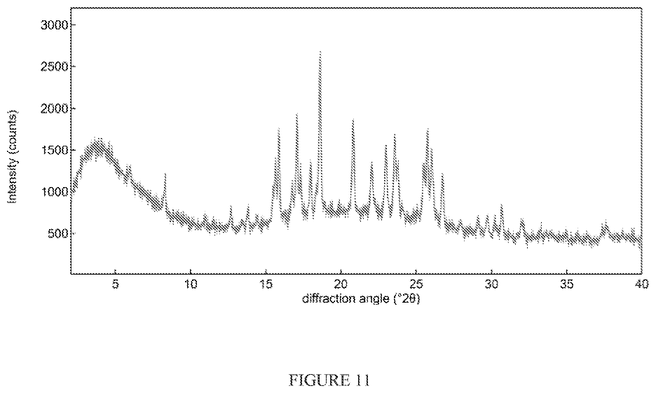

Crystalline Form F was characterized using various techniques which are described in further detail in the experimental methods section. FIG. 11 shows the crystalline structure of Form E as determined by X-ray powder diffraction (XRPD). Crystalline Form E, which may be obtained by the methods disclosed herein, exhibits prominent peaks at approximately 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ. Thus, in some embodiments, a crystalline form of Compound 1 has at least one characteristic peak (e.g., one, two, three, four, five, six, seven, eight, nine, ten, eleven, twelve, thirteen, fourteen, fifteen, sixteen, or seventeen characteristic peaks) selected from approximately 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ. In some embodiments, a crystalline form of Compound 1 has at least three characteristic peaks selected from approximately 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ.

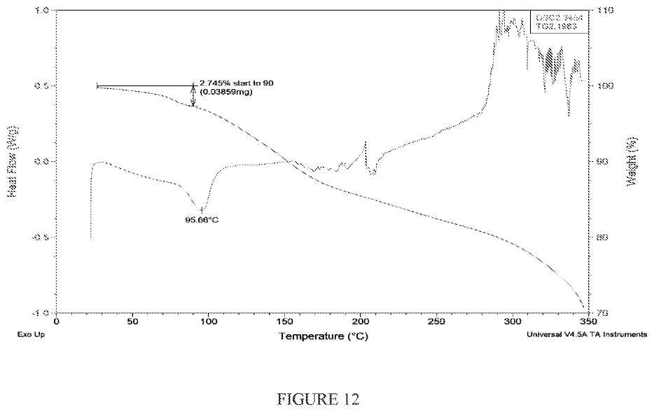

FIG. 12 shows results obtained by differential scanning calorimetry (DSC) and thermogravimetric analysis for crystalline Form F. The DSC results indicate an endotherm at 96° C. for crystalline Form F. Crystalline Form F was analysed by thermogravimetric gravimetric analysis (TG), and in one instance exhibited a 2.75% weight loss when carried out from 25° C. to 90° C. ¹H-NMR results (not shown) indicate the formation of a dimethylformamide solvate.

### Compound 1-A and Crystalline Forms Thereof

Disclosed herein is Compound 1-A, which is the (2-dimethylamino)ethanol (“deanol”) salt of Compound 1. The structure of Compound 1-A is provided below.

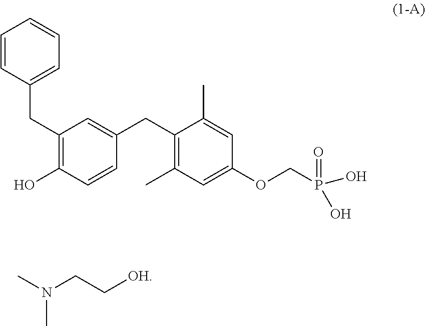

Also disclosed herein are crystalline forms of Compound 1-A, and in particular crystalline Form 1, crystalline Form 2, crystalline Form 3, and crystalline Form 4. (described below). Unless otherwise stated, the X-ray powder diffraction data provided herein was determined using a Cu Kα radiation source.

### Crystalline Form 1

Some embodiments include an unsolvated crystalline form of Compound 1-A, referred to herein as crystalline Form 1. The precise conditions for forming crystalline Form 1 may be empirically determined and it is only possible to give a number of methods which have been found to be suitable in practice.

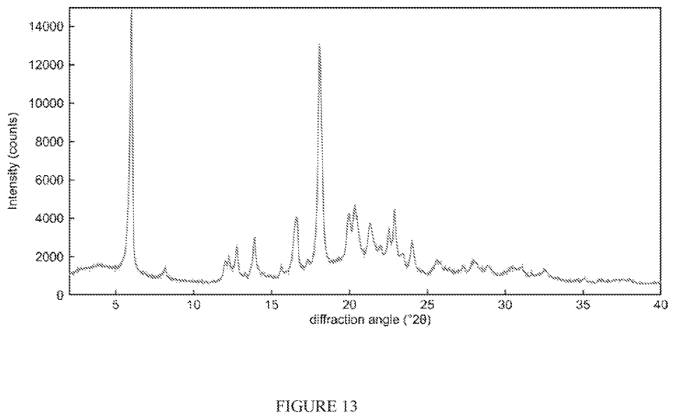

Crystalline Form 1 was characterized using various techniques which are described in further detail in the experimental methods section. FIG. 13 shows the crystalline structure of Form 1 as determined by X-ray powder diffraction (XRPD). Crystalline Form 1, which may be obtained by the methods disclosed herein, exhibits prominent peaks at approximately 6.1, 12.2, 13.3, 13.6, 16.5, 17.3, 18.3, 19.9, 20.2, 20.5, 21.4, 21.9, 22.5, 22.8, 23.0, and 25.6 degrees 2θ. Thus, in some embodiments, a crystalline form of Compound 1-A has at least one characteristic peak (e.g., one, two, three, four, five, six, seven, eight, nine, ten, eleven, twelve, thirteen, fourteen, fifteen, or sixteen characteristic peaks) selected from approximately 6.1, 12.2, 13.3, 13.6, 16.5, 17.3, 18.3, 19.9, 20.2, 20.5, 21.4, 21.9, 22.5, 22.8, 23.0, and 25.6 degrees 2θ. In some embodiments, a crystalline form of Compound 1-A has at least three characteristic peaks selected from approximately 6.1, 12.2, 13.3, 13.6, 16.5, 17.3, 18.3, 19.9, 20.2, 20.5, 21.4, 21.9, 22.5, 22.8, 23.0, and 25.6 degrees 2θ.

DVS results (not shown) indicate that Crystalline Form 1 is moderately hygroscopic. DSC analysis indicates a melting point of about 139° C. for crystalline Form 1.

### Crystalline Form 2

Some embodiments include an unsolvated crystalline form of Compound 1-A, referred to herein as crystalline Form 2. The precise conditions for forming crystalline Form 1 may be empirically determined and it is only possible to give a number of methods which have been found to be suitable in practice.

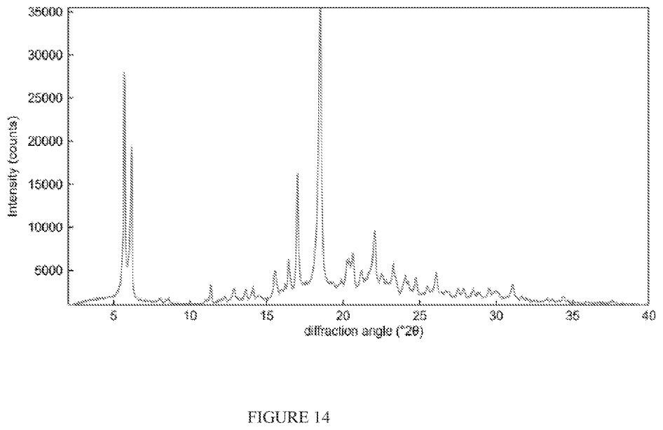

Crystalline Form 2 was characterized using various techniques which are described in further detail in the experimental methods section. FIG. 14 shows the crystalline structure of Form 2 as determined by X-ray powder diffraction (XRPD). Crystalline Form 2, which may be obtained by the methods disclosed herein, exhibits prominent peaks at approximately 5.7, 6.2, 15.5, 16.4, 17.0, 18.5, 20.6, 21.2, 22.0, 23.3, and 26.1 degrees 2θ. Thus, in some embodiments, a crystalline form of Compound 1-A has at least one characteristic peak (e.g., one, two, three, four, five, six, seven, eight, nine, ten, or eleven characteristic peaks) selected from approximately 5.7, 6.2, 15.5, 16.4, 17.0, 18.5, 20.6, 21.2, 22.0, 23.3, and 26.1 degrees 2θ. In some embodiments, a crystalline form of Compound 1-A has at least three characteristic peaks selected from approximately 5.7, 6.2, 15.5, 16.4, 17.0, 18.5, 20.6, 21.2, 22.0, 23.3, and 26.1 degrees 2θ.

DVS results (not shown) indicate that Crystalline Form 2 is moderately hygroscopic. DSC analysis indicates a melting point of about 117° C. for crystalline Form 2.

### Crystalline Form 3

Some embodiments include a crystalline form of Compound 1-A, referred to herein as crystalline Form 3. The precise conditions for forming crystalline Form 3 may be empirically determined and it is only possible to give

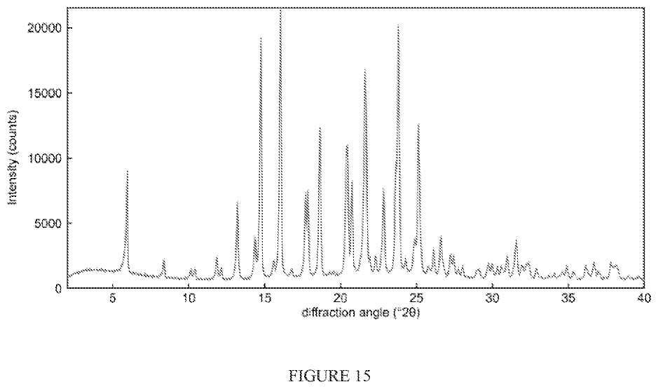

Crystalline Form 3 was characterized using various techniques which are described in further detail in the experimental methods section. FIG. 15 shows the crystalline structure of Form 3 as determined by X-ray powder diffraction (XRPD). Crystalline Form 3, which may be obtained by the methods disclosed herein, exhibits prominent peaks at approximately 6.0, 13.3, 14.4, 14.8, 16.1, 17.8, 17.9, 18.7, 20.5, 20.8, 21.7, 22.9, 23.8, 24.9, 25.1, and 26.6 degrees 2θ. Thus, in some embodiments, a crystalline form of Compound 1-A has at least one characteristic peak (e.g., one, two, three, four, five, six, seven, eight, nine, ten, eleven, twelve, thirteen, fourteen, fifteen, or sixteen characteristic peaks) selected from approximately 6.0, 13.3, 14.4, 14.8, 16.1, 17.8, 17.9, 18.7, 20.5, 20.8, 21.7, 22.9, 23.8, 24.9, 25.1, and 26.6 degrees 2θ. In some embodiments, a crystalline form of the compounds of Formula (I) has at least three characteristic peaks selected from approximately 6.0, 13.3, 14.4, 14.8, 16.1, 17.8, 17.9, 18.7, 20.5, 20.8, 21.7, 22.9, 23.8, 24.9, 25.1, and 26.6 degrees 2θ.

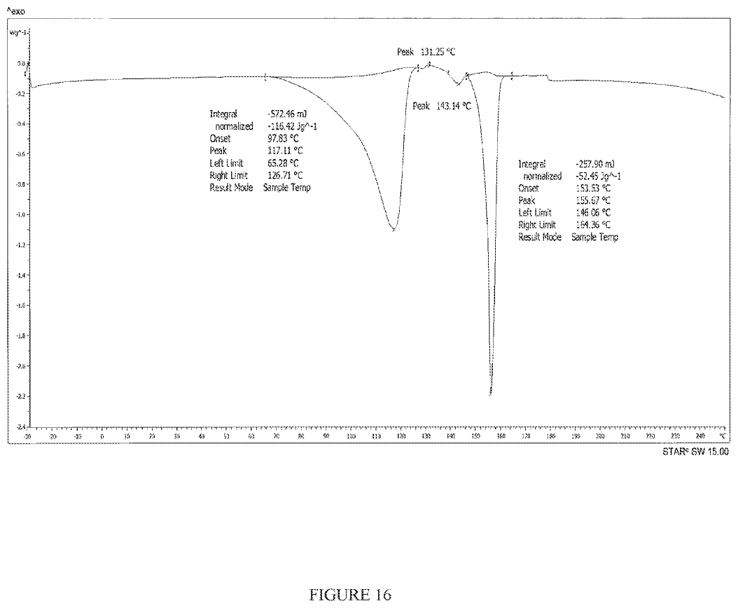

FIG. 16 shows DSC and TGA results for crystalline Form 3. The DSC results show two endotherms: the first at a temperature of about 117° C. indicating the loss of water and 156° C., which indicates the melting point for the crystal. Accordingly, in some embodiments, crystalline Form 3 exhibits a melting point from about about 148° C., 152° C., or at about 156° C. In some embodiments, crystalline Form 3 may have a melting point at from about 146° C. to about 158° C., or at from about 148° C. to about 156° C. In some embodiments, crystalline Form 3 may have a DSC melting point at from about 146° C. to about 150° C. or at from about 152° C. to about 156° C. ¹H-NMR results are consistent with a 1:1 molar salt of Compound 1 and deanol. Crystalline Form 3 was analyzed by thermogravimetric gravimetric analysis (TG), and in one instance exhibited a 3.85% weight loss when carried out from 25° C. to 90° C. and a 2.09% weight loss when carried out from 90° C. to 150° C. DVS data (not shown) indicates slight water uptake. Karl Fisher titration indicates that Crystalline form 3 is a monohydrate.

### Crystalline Form 4

Some embodiments include an unsolvated crystalline form of Compound 1-A, referred to herein as crystalline Form 4. The precise conditions for forming crystalline Form 4 may be empirically determined and it is only possible to give a number of methods which have been found to be suitable in practice.

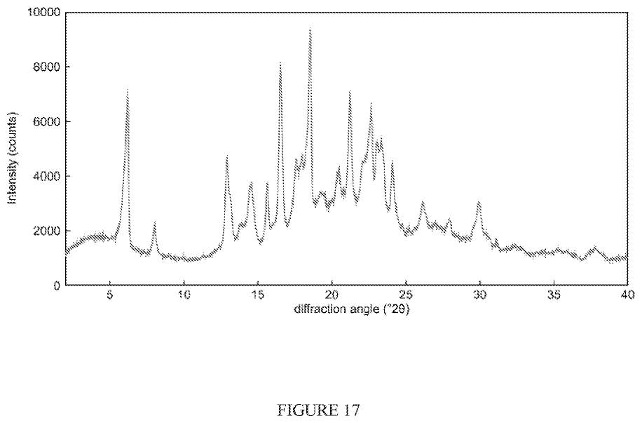

Crystalline Form 4 was characterized using various techniques which are described in further detail in the experimental methods section. FIG. 17 shows the crystalline structure of Form 4 as determined by X-ray powder diffraction (XRPD). Crystalline Form 4, which may be obtained by the methods disclosed herein, exhibits prominent peaks at approximately 6.2, 12.9, 14.0, 14.5, 16.5, 17.6, 18.0, 18.6, 20.3, 21.2, 22.7, 23.3, 24.0, and 26.1 degrees 2θ. Thus, in some embodiments, a crystalline form of Compound 1-A has at least one characteristic peak (e.g., one, two, three, four, five, six, seven, eight, nine, ten, eleven, twelve, thirteen, or fourteen characteristic peaks) selected from approximately 6.2, 12.9, 14.0, 14.5, 16.5, 17.6, 18.0, 18.6, 20.3, 21.2, 22.7, 23.3, 24.0, and 26.1 degrees 2θ. In some embodiments, a crystalline form of the compounds of Formula (I) has at least three characteristic peaks selected from approximately 6.2, 12.9, 14.0, 14.5, 16.5, 17.6, 18.0, 18.6, 20.3, 21.2, 22.7, 23.3, 24.0, and 26.1 degrees 2θ.

FIG. 17 shows DSC and TGA results for crystalline Form 4. The DSC results an endotherm at 114° C., which indicates the melting point for the crystal. Accordingly, in some embodiments, crystalline Form 4 exhibits a melting point from about 111° C. to 117° C., from about 106° C. to about 116° C., or at about 114° C. ¹H-NMR results are consistent with a 1:1 molar salt of Compound 1 and deanol. Crystalline Form 4 was analyzed by thermogravimetric gravimetric analysis (TG), and in one instance exhibited a 0.44% weight loss when carried out from 25° C. to 120° C.

### Methods of Crystalizing Compound 1 and 1-A

Disclosed are methods of crystalizing Compound 1. Crystalline forms of Compound 1 may generally be obtained or produced by crystallizing Compound 1 under controlled conditions. In some embodiments, the method may produce an unsolvated crystalline form. In some embodiments, the method may produce a solvated crystal form. In some embodiments, the method may produce the crystalline Form A. In some embodiments, the method may produce the crystalline Form B. In some embodiments, the method may produce the crystalline Form C. In some embodiments, the method may produce the crystalline Form D. In some embodiments, the method may produce the crystalline Form E. In some embodiments, the method may produce the crystalline Form F. In some embodiments, the method may produce a mixture of any of the aforementioned crystalline forms.

Also disclosed are methods of crystalizing Compound 1-A. Crystalline forms of Compound 1 may generally be obtained or produced by crystallizing Compound 1-A under controlled conditions. In some embodiments, the method may produce an unsolvated crystalline form. In some embodiments, the method may produce a solvated crystal form. In some embodiments, the method may produce the crystalline Form 1. In some embodiments, the method may produce the crystalline Form 2. In some embodiments, the method may produce the crystalline Form 3. In some embodiments, the method may produce the crystalline Form 4. In some embodiments, the method may produce a mixture of any of the aforementioned crystalline forms.

In some embodiments, the method may comprise dissolving an amorphous form of Compound 1 or Compound 1-A in a first solvent to create a first solution. In some embodiments, the method may comprise dissolving a crystalline form of Compound 1 or Compound 1-A in a first solvent to create a first solution. In some embodiments, the method may comprise dissolving a mixture of amorphous and crystalline forms of Compound 1 or Compound 1-A in a first solvent to create a first solution. In some embodiments, the method may comprise adding a second solvent to the first solution to create a second mixture. In some embodiments, the method may comprise evaporating the second mixture. In some embodiments, the method may comprise cooling the second mixture. In some embodiments, the second mixture may be cooled to 25° C., 20° C., 15° C., 10° C., 5° C., 0° C., −5° C., −10° C., −20° C., −25° C., any range between any of these values, or below −25° C.

In some embodiments, the method may comprise isolating the crystalline form of Compound 1 or Compound 1-A. In some embodiments, isolation may performed by filtration, such as hot-filtration. In some embodiments, the isolated product may be dried, such as by air drying.

In some embodiments, the first solvent may be a single solvent. In some embodiments, the first solvent may be a mixture of two or more solvents. In some embodiments, the first solvent may be acetone, acetonitrile, 1,4-dioxane, dimethylformamide, ethanol, ethyl acetate, diethyl ether, methanol, methyl ethyl ketone, 2-methyltetrahydrofuran, isopropanol, n-propanol, isopropyl acetate, tetrahydrofuran, water, or a mixture thereof.

In some embodiments, the second solvent may be a single solvent. In some embodiments, the second solvent may be a mixture of two or more solvents. In some embodiments, the first solvent may comprise acetone, acetonitrile, 1,4-dioxane, dimethylformamide, ethanol, ethyl acetate, diethyl ether, methanol, methyl ethyl ketone, 2-methyltetrahydrofuran, isopropanol, n-propanol, isopropyl acetate, tetrahydrofuran, water, or a mixture thereof. In some embodiments, the second solvent may comprise toluene, hexanes, water, dichloromethane, or a combination thereof.

In some embodiments, the method may further comprise agitation. In some embodiments, agitation may be performed by stirring. In some embodiments, agitation may be performed by sonication.

In some embodiments, portions of the method may performed at the same temperature. In some embodiments, portions of the method may be performed at various temperatures. In some embodiments, portions of the method may be performed at room temperature. In some embodiments, portions of the method may be performed at −40° C. to 200° C. In some embodiments, portions of the method may be performed at −40° C. to 25° C. In some embodiments, portions of the method may be performed at −25° C. to −10° C. In some embodiments, portions of the method may be performed at 2° C. to 8° C. In some embodiments, portions of the method may be performed at 50° C. to 60° C. In some embodiments, portions of the method may be performed at 65° C. to 75° C. In some embodiments, portions of the method may be performed at 75° C. to 150° C. In some embodiments, portions of the method may include the first solution, second mixture, seeded mixture, isolation of the crystalline form, and agitation.

In some embodiments, the crystalline form of Compound 1-A may be prepared by dissolving a crystalline or amorphous form of Compound 1 in a first solvent to create a first solution; dissolving 1 molar equivalent of (2-dimethylamino)ethanol in a second solvent to create a second solution; combining the first solution and the second solution to form a combined solution. In some embodiments the first solvent may comprise acetone, acetonitrile, 1,4-dioxane, dimethylformamide, ethanol, ethyl acetate, diethyl ether, methanol, methyl ethyl ketone, 2-methyltetrahydrofuran, isopropanol, n-propanol, isopropyl acetate, tetrahydrofuran, water, or a mixture thereof. In some embodiments, the second solvent may comprise acetone, acetonitrile, 1,4-dioxane, dimethylformamide, ethanol, ethyl acetate, diethyl ether, methanol, methyl ethyl ketone, 2-methyltetrahydrofuran, isopropanol, n-propanol, isopropyl acetate, tetrahydrofuran, water, or a mixture thereof. In some embodiments, the first solvent and second solvent may the same. In some embodiments, the first solvent and second solvent may be different. In some embodiments, the combined solution may be further agitated, evaporated, and/or cooled as described herein.

In some embodiments, crystalline Form A of Compound 1 may be prepared by dissolving a quantity of Compound 1 in a minimal solvent at a first temperature to form a first solution. In some embodiments, the solvent may be acetonitrile. In other embodiments, the solvent may be ethyl acetate. In yet other embodiments, the solvent may be acetone. In still yet other embodiments, the solvent may be diethyl ether. In other embodiments the solvent may by methyl ethyl ketone. In some embodiments, the first temperature is from 40° C. to 60° C. or 45° C. to 55° C. In some specific embodiments, the first temperature is about 50° C. In some embodiments, the first solvent may be cooled to a second temperature. In some embodiments, the second temperature may be −5° C. to −25° C. or −10° C. to −20° C. In other embodiments, the second temperature may be room temperature. In some specific embodiments, the second temperature is about −15° C. In some embodiments, an antisolvent may be added to the first solution prior to cooling. In some embodiments, the antisolvent is hexane. In other embodiments, the antisolvent is toluene.

In some embodiments, crystalline Form A of Compound 1 may be prepared by combining a quantity of Compound 1 in a solvent at room temperature and milling the solid. In some embodiments the quantity of Compound 1 is 20 mg and the quantity of solvent is 10 μL. In some embodiments, the solvent is acetone, acetonitrile, ethanol, ethyl acetate, methanol, methyl ethyl ketone, tetrahydrofuran, or water.

In some embodiments, crystalline Form A of Compound 1 may be prepared by adding Compound 1 to a solvent to form a slurry. In some embodiments, the solvent is dichloromethane. In other embodiments, the solvent is water. In some embodiment the slurry may be stirred at 20, 25, 30, 35, 40, 45, or 50° C. In some embodiments, the slurry may be stirred for 1, 2, 3, 4, 5, 6, or 7 days or more.

In some embodiments, crystalline Form B of Compound 1 may be prepared by combining a quantity of Compound 1 in a solvent at room temperature and milling the solid. In some embodiments the quantity of Compound 1 is 20 mg and the quantity of solvent is 10 μL. In some embodiments, the solvent is isopropanol.

In some embodiments, crystalline Form C of Compound 1 may be prepared by dissolving a quantity of Compound 1 in a minimal amount solvent at room temperature leaving the solid to evaporate. In some embodiments, the solvent is acetone, acetonitrile, 1,4-dioxane, ethanol, methanol, isopropanol, water, or a mixture thereof.

In some embodiments, crystalline Form D of Compound 1 may be prepared by dissolving a quantity of Compound 1 in a minimal solvent at a first temperature to form a first solution. In some embodiments, the solvent may be acetone. In other embodiments, the solvent may be tetrahydrofuran. In yet other embodiments, the solvent may be ethanol. In still yet other embodiments, the solvent may be water. In other embodiments the solvent may be methanol/water (95/5 v/v). In some embodiments, the first temperature is from 40° C. to 60° C. or 45° C. to 55° C. In some specific embodiments, the first temperature is about 50° C. In some specific embodiments, the first temperature is about 60° C. In some embodiments, the first solvent may be cooled to a second temperature. In some embodiments, the second temperature may be −5° C. to −25° C. or −10° C. to −20° C. In other embodiments, the second temperature may be room temperature. In some specific embodiments, the second temperature is about −15° C. In some embodiments, an antisolvent may be added to the first solution prior to cooling. In some embodiments, the solvent is THF and the antisolvent is water. In some embodiments, the solvent is THF and the antisolvent is hexane. In some embodiments, the solvent is THF and the antisolvent is dichloromethane.

In some embodiments, crystalline Form E of Compound 1 may be prepared by dissolving a quantity of Compound 1 in a minimal solvent at a first temperature to form a first solution. In some embodiments, the solvent may be acetonitrile and water 95:5 (v/v). In other embodiments, the solvent may be methanol. In some embodiments, the solvent is diethyl ether. In other embodiments, the solvent is methyl ethyl ketone. In some embodiments, the solvent may be methanol. In some embodiments, the first temperature is from 40° C. to 60° C. or 45° C. to 55° C. In some specific embodiments, the first temperature is about 50° C. In some specific embodiments, the first temperature is about 60° C. In some embodiments, the first solvent may be cooled to a second temperature. In some embodiments, the second temperature may be −5° C. to −25° C. or −10° C. to −20° C. In other embodiments, the second temperature may be room temperature. In some specific embodiments, the second temperature is about −15° C. In some embodiments, an antisolvent may be added to the first solution prior to cooling. In some embodiments, the solvent is methanol and the antisolvent is toluene. In some embodiments, the solvent is isopropanol and the antisolvent is hexane. In some embodiments, the solvent is diethyl ether and the antisolvent is dichloromethane. In some embodiments, the solvent is methanol and the antisolvent is dichloromethane. In some embodiments, the solvent is methyl ethyl ketone and the antisolvent is dichloromethane.

In some embodiments, crystalline Form F of Compound 1 may be prepared by dissolving a quantity of Compound 1 in a minimal amount solvent at room temperature leaving the solid to evaporate. In some embodiments, the solvent is dimethylformamide.

In some embodiments, crystalline Form F of Compound 1 may be prepared by dissolving a quantity of Compound 1 in a minimal solvent at a first temperature to form a first solution. In some embodiments, the solvent may be dimethylformamide. In some embodiments, the first temperature is from 40° C. to 60° C. or 45° C. to 55° C. In some specific embodiments, the first temperature is about 50° C. In some embodiments, the first solvent may be cooled to a second temperature. In some embodiments, the second temperature may be −5° C. to −25° C. or −10° C. to −20° C. In some specific embodiments, the second temperature is about −15° C. In some embodiments, an antisolvent may be added to the first solution prior to cooling. In some embodiments, the antisolvent is toluene.

In some embodiments, crystalline Form 1 of Compound 1-A may be prepared by combining Compound 1 with a solvent to form a first solution and adding said solution to 1 molar equivalent of (2-methylamino)ethanol to form a second solution. The second solution may be stirred at room temperature for 1, 2, 3, 4, 5, or more days. In some embodiments, the first solution may be sonicated prior to addition to (2-methylamino)ethanol. In some embodiments, the solvent is acetone. In other embodiments, the solvent is 1,4-dioxane. In yet other embodiments, the solvent is diethyl ether. In some embodiments, the solvent is isopropanol. In other embodiments, the solvent is tetrahydrofuran. In some embodiments, the solvent is methyl ethyl ketone.

In some embodiments, crystalline Form 3 of Compound 1-A may be prepared by combining Compound 1 in a first solvent to form a first solution. In some embodiments, the first solvent may be acetone. In some embodiments, the acetone may contain 0.5%, 1.0%, 1.5%, 2.0%, 2.5%, 3.0%, 3.5%, 4.0%, 4.5%, or 5.0% water. In some embodiments, the first solution may be added to a second solution of 2-(dimethylamino)ethanol in a second solvent to form a third solution. In some embodiments, the second solvent may be acetone. In some embodiments, the acetone may contain 0.5%, 1.0%, 1.5%, 2.0%, 2.5%, 3.0%, 3.5%, 4.0%, 4.5%, or 5.0% water. In some embodiments the molar ratio of 2-(dimethylamino)ethanol to Compound 1-A is 1:1, 1.05:1, 1.1:1, 1.2:1, 1.3:1, 1.4:1, or 1:5 to 1. In some embodiments, molar ratio of 2-(dimethylamino)ethanol to Compound 1-A is from 1:1 to 1.1:1. In some specific embodiments, molar ratio of 2-(dimethylamino)ethanol to Compound 1-A is 1.05:1. In some embodiments, the first solution may be added to the second solution over a period of 5, 10, 15, 20, 25, or 30 minute or more. In some embodiments, a slurry is formed upon addition of the first solution to the second solution. In some embodiments, additional solvent may be added to the slurry. In some embodiments, the additional solvent is acetone. In some embodiments, the acetone may contain 0.5%, 1.0%, 1.5%, 2.0/0, 2.5%, 3.0%, 3.5%, 4.0%, 4.5%, or 5.0% water

In some embodiments, crystalline Form 3 of Compound 1-A may be prepared by combining Compound 1 with 1 molar equivalent of (2-methylaminio)ethanol pre-dissolved in ethanol form a solution. In some embodiments, the solution may be sonicated and allowed to evaporate until solids form. In some embodiments, the solution may be stirred at room temperature for 1, 2, 3, 4, 5, or more days.

In some embodiments, crystalline Form 4 of Compound 1-A may be prepared by combining Compound 1 with a solvent to form a first solution and adding said solution to 1 molar equivalent of (2-methylaminio)ethanol to form a second solution. The second solution may be stirred at room temperature for 1, 2, 3, 4, 5, or more days. In some embodiments, the first solution may be sonicated prior to addition to (2-methylaminio)ethanol. In some embodiments, the solvent is acetonitrile or ethyl acetate.

### Definitions

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Disease or condition in a subject” means a subject exhibiting one or more clinical indicators of a disease or condition. In certain embodiments, the disease or condition is one or more fibroses, fibrotic conditions, or fibrotic symptoms. In certain embodiments, the disease or condition is scleroderma. In certain embodiments, the disease or condition is non-alcoholic steatohepatitis (NASH). In certain embodiments, the disease or condition is cirrhosis. In certain embodiments, the disease or condition is non-alcoholic fatty liver disease (NAFLD). In certain embodiments, the disease or condition is idiopathic pulmonary fibrosis. In certain embodiments, the disease or condition is atherosclerosis. In certain embodiments, the disease or condition is hepatitis, alcoholic fatty liver disease, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, diffuse parenchymal lung disease, idiopathic interstitial fibrosis, diffuse interstitial fibrosis; interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components.

As used herein, “fibrosis” refers to the abnormal deposition of extracellular matrix proteins. Such proteins include but are not limited to collagen, elastin, fibronectin, laminin, keratin, keratin, keratin sulfate, fibrin, perlecan, agrin, or aggrecan. As used herein, “collagen” refers to any one of the subtypes of collagen, including but not limited to Type I, I, III, IV, V, VI, VII, VIII, IX, X, XI, XII, XIII, XIV, XV, XVI, XVII, or XVIII. Exemplary collagen types and subtypes especially include Type I, Type Ia, Type II, Type III, Type IV, and Type V. As used herein, fibrosis may occur by itself or as a symptom or sequela of another condition. As used herein, fibrosis may result from a genetic condition, a genetic predisposition, an environmental insult, an injury, healing of an injury, an autoimmune condition, or a chronic inflammation, a chronic inflammatory condition, or another condition leading to abnormal or excessive deposition of extracellular matrix components. Fibrosis as referred to herein may be assessed by assaying for, or determining the presence or level of, one or more biomarkers. Biomarkers for the presence of fibrosis include, but are not limited to, expression of the Col1a1, Col3a1, ACTA2, ENPP2, and/or LGALS1 genes or any combination or product thereof. Diagnosis or assessment of fibrosis may further be made by determination of the presence or level of type I collagen and/or hydroxyproline or any combination or product thereof. Diagnosis or assessment of fibrosis may also be made by histological, histochemical, or immunohistochemical analysis of one or more samples from a subject.

“Glycogen storage disease” means any one or more of a group of disorders marked by dysfunction in the synthesis, transport, or utilization of glycogen, generally due to the loss of a necessary enzyme activity. Glycogen storage diseases are generally classified by type according to their symptoms and etiologies. Known types include GSD type 0 (aglycogenesis, glycogen synthase deficiency); GSD type 1 (von Gierke disease, glucose-6-phosphatase translocase/transporter deficiency, GSD I); GSD type 2 (Pompe disease, alpha-1-4-glucosidase deficiency, GSD II); GSD type 3 (Cori disease, Forbes disease, limit dextrinosis, debranching enzyme disease; amylo-1-6-glucosidase deficiency due to loss of glucosidase, and/or transferase activity, GSD III); GSD type 4 (Andersen disease, glycogen phosphorylase deficiency, brancher deficiency, amylopectinosis, glycogen branching enzyme deficiency; amylo-1,4 to 1,6 transglucosidase deficiency, GSD IV); GSD type 5 (McArdle disease; glycogen phosphorylase (muscle type) deficiency, GSD V); GSD type 6 (Hers disease; glycogen phosphorylase E (liver type) deficiency, GSD V1), GSD type 7 (Tarui disease; phosphofructokinase deficiency, GSD VII); GSD type 8, 9 (GSD with phosphorylase activation system defects; phosphorylase kinase (liver or muscle isoforms) deficiency, GSD VIII and GSD IX); GSD type 10 (cyclic AMP-dependent kinase deficiency, GSD X), GSD type 11 (Fanconi-Bickel syndrome; glucose transporter type 2 (GLUT2) deficiency, GSD XI); and GSD type 12 (aldolase A deficiency, GSD XII). Subtypes of glycogen storage diseases are also known, in particular GSD 1a, which results from mutations in the gene for glucose-6-phosphatase (G6PC) and leads to, among other symptoms, the excess accumulation of glycogen and lipids in liver tissue, hepatomegaly, hepatic adenomas, and hepatocellular carcinoma. Symptoms of glycogen storage diseases may include elevated or reduced blood sugar, insulin insensitivity, myopathies, as well as hepatic symptoms such as steatosis, hyperlipidemia, hypercholesterolemia, cardiomegaly, hepatomegaly, fibrosis, cirrhosis, hepatocellular adenoma, and hepatocellular carcinoma. Symptoms may also include insulin insensitivity, elevated or reduced blood glucose, renal dysfunction, and/or fibrosis.

As used herein, “inflammatory disease” refers to a disease or disorder that is characterized by inflammation. Exemplary inflammatory diseases include, but are not limited to, acne, acid reflux/heartburn, age related macular degeneration (AMD), allergy, allergic rhinitis, Alzheimer's disease, amyotrophic lateral sclerosis, anemia, appendicitis, arteritis, arthritis, asthma, atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD), cirrhosis, colitis, congestive heart failure, conjunctivitis, cyclophosphamide-induced cystitis, cystic fibrosis, cystitis, common cold, dacryoadenitis, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, inflammatory neuropathy, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), a migraine, multiple sclerosis, myelitis, myocarditis, myositis, nephritis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peritonitis, pharyngitis, phlebitis, pleuritis, pneumonitis, polycystic nephritis, proctitis, prostatitis, psoriasis, pulpitis, pyelonephritis, pylephlebitis, renal failure, reperfusion injury, retinitis, rheumatic fever, rhinitis, salpingitis, sarcoidosis, sialadenitis, sinusitis, spastic colon, stenosis, stomatitis, stroke, surgical complication, synovitis, tendonitis, tendinosis, tenosynovitis, thrombophlebitis, tonsillitis, trauma, traumatic brain injury, transplant rejection, trigonitis, tuberculosis, tumor, urethritis, ursitis, uveitis, vaginitis, vasculitis, and vulvitis. Inflammation as referred to herein may be assessed by assaying for, or determining the presence or level of, one or more biomarkers. Biomarkers for the presence of inflammation include, but are not limited to, expression of the TNF, CARD15, IL4R, IL23R, CTLA4, ANXA1, ANXA2, LGALS3, and/or PTPN22 genes or any combination or product thereof.

The term “atherosclerosis” refers to a condition characterized by irregularly distributed lipid deposits in the intima of large and medium-sized arteries wherein such deposits provoke fibrosis and calcification. Atherosclerosis raises the risk of angina, stroke, heart attack, or other cardiac or cardiovascular conditions.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Solvate” refers to the compound formed by the interaction of a solvent and an EPI, a metabolite, or salt thereof. Suitable solvates are pharmaceutically acceptable solvates including hydrates.

### Pharmaceutical Compositions

Compound 1 and Compound 1-A and crystal forms thereof described herein can be formulated into pharmaceutical compositions for use in treatment of the conditions described herein. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

In some embodiments, the crystal forms of Compound 1 and Compound 1-A described herein may be formulated into a single pharmaceutical composition for use in treatment of the conditions described herein. In some embodiments, a formulation comprising the Compound 1 and/or Compound 1-A and crystal forms thereof described herein may be administered in combination with one or more second pharmaceutical agents.

In some embodiments, Compound 1 and/or Compound 1-A, and crystal forms thereof described herein may be dissolved in a solvent prior to administration to a subject in need thereof.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002), Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, Compound 1 and/or Compound 1-A, and crystal forms thereof described herein and compositions thereof described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J. Pharm. Sci. Tech. 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of Compound 1 and/or Compound 1-A, and crystal forms thereof described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, t Compound 1 and/or Compound 1-A, and crystal forms thereof described herein is administered at a dose in the range of about 1-50 mg/m² of the body surface area. In some embodiments, Compound 1 and/or Compound 1-A, and crystal forms thereof described herein may be administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, Compound 1 and/or Compound 1-A, and crystal forms thereof described herein may be administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 and Compound 1-A, and crystal forms thereof described herein may be administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 and/or Compound 1-A, and crystal forms thereof described herein may be administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the dose of Compound 1 and/or Compound 1-A, and crystal forms thereof described herein may be about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the dose of Compound 1 and/or Compound 1-A, and crystal forms thereof described herein may be about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the dose of Compound 1 and/or Compound 1-A, and crystal forms thereof described herein may be about 20 mg-60 mg, 27 mg-60 mg, 20 mg-45 mg, or 27 mg-45 mg. In some embodiments, the dose of the crystal forms of Compound 1 and Compound 1-A described herein may be about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the dose of Compound 1 and/or Compound 1-A, and crystal forms thereof described herein may be greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the dose of Compound 1 and/or Compound 1-A, and crystal forms thereof described herein may be less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the dose of Compound 1 and/or Compound 1-A, and crystal forms thereof described herein may be about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

Compound 1 and/or Compound 1-A, and crystal forms thereof described herein may also be incorporated into formulations for delivery outside the systemic circulation. Such formulations may include enteric-coated capsules, tablets, soft-gels, spray dried powders, polymer matrices, hydrogels, enteric-coated solids, crystalline solids, amorphous solids, glassy solids, coated micronized particles, liquids, nebulized liquids, aerosols, or microcapsules.

### Methods of Administration

The compositions described above may be administered through any suitable route of administration, for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouthrinse; nasally, such as by a nasal aerosol, powder, or spray; or ocularly, such as by an eye drop). In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, Compound 1 and/or Compound 1-A, and crystal forms thereof described herein may be administered simultaneously with one or more second pharmaceutical agents. In other embodiments, Compound 1 and/or Compound 1-A, and crystal forms thereof described herein may be administered sequentially with one or more second pharmaceutical agents.

### Methods of Treatment

Some embodiments according to the methods and compositions of the present disclosure relate to a method for preventing, treating, or ameliorating one or more fatty liver diseases or disorders in a subject comprising administering an effective amount of Compound 1 and/or Compound 1-A, and crystal forms thereof described herein to a subject in need thereof. In some embodiments, the disease or disorder may be a fatty liver disease. In some embodiments, the fatty liver disease may be steatosis. In other embodiments, the fatty liver disease may be non-alcoholic fatty liver disease. In some embodiments, the fatty liver disease may be non-alcoholic steatohepatitis (NASH). In some embodiments, the subject may have two or more of the aforementioned fatty liver diseases.

Some embodiments according to the methods and compositions of the present disclosure relate to a method for the reduction or prevention of the deposition of extracellular matrix proteins, comprising administering an effective amount of Compound 1 and/or Compound 1-A, and crystal forms thereof described herein to a subject in need thereof. In some embodiments, said deposition of extracellular matrix proteins may comprise abnormal or excessive deposition of said proteins. In some embodiments, said extracellular matrix proteins may comprise one or more of collagen, keratin, elastin, or fibrin. In some embodiments, said extracellular matrix proteins may comprise collagen. In some embodiments, said extracellular matrix proteins may comprise Type I collagen. In some embodiments, said extracellular matrix proteins may comprise Collagen Type Ia. In some embodiments, said extracellular matrix proteins may comprise Type III collagen. Some embodiments according to the compositions and methods of the present disclosure relate to a method for the treatment of a fibrosis or its symptoms or sequelae, comprising administering an effective amount of Compound 1 and/or Compound 1-A, and crystal forms thereof described herein to a subject in need thereof.

In some embodiments, the compounds and compositions Compound 1 and/or Compound 1-A, and crystal forms thereof described herein can be used in a method of preventing, treating, or ameliorating one or more diseases or disorders in a subject, comprising administering a compound disclosed herein, or a pharmaceutically acceptable salt thereof, to a subject in need thereof. In some embodiments, the disease or disorder may be liver fibrosis, renal fibrosis, biliary fibrosis, pancreatic fibrosis, nonalcoholic steatohepatitis, non-alcoholic fatty liver disease, chronic kidney disease, diabetic kidney disease, primary sclerosing cholangitis, primary biliary cirrhosis, or idiopathic fibrosis. In some embodiments, the disease or disorder may nonalcoholic steatohepatitis, non-alcoholic fatty liver disease, chronic kidney disease, diabetic kidney disease, primary sclerosing cholangitis, or primary biliary cirrhosis.

In some embodiments, the compounds and compositions comprising a crystal form of Compound 1 and/or Compound 1-A described herein can be used to treat a variety of conditions arising from fibrosis or inflammation, and specifically including those associated with abnormal collagen deposition. Example conditions include glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen.

In some embodiments the methods of the present disclosure comprise methods for the treatment, amelioration, or prevention of a fibrotic condition. In some embodiments, said fibrotic condition may be secondary to another condition. In some embodiments, said fibrotic condition or primary condition may further comprise chronic inflammation of an organ, tissue, spatial region, or fluid-connected area of the body of a subject. In some embodiments, said inflammation may comprise activation of one or more TGF-beta dependent signaling pathways. In some embodiments, said TGF-β dependent signaling pathways may comprise one or more elements responsive to T3 or T4. In some embodiments, said fibrotic condition may comprise abnormal or excessive deposition of one or more of collagen, keratin, or elastin. In some embodiments, said fibrotic condition may comprise abnormal or excessive deposition of collagen. In some embodiments, said fibrotic condition may comprise abnormal or excessive deposition of Type I collagen. In some embodiments, said fibrotic condition may comprise abnormal or excessive deposition of Collagen Type Ia. In some embodiments, said fibrotic condition may comprise abnormal or excessive deposition of Type III collagen. In some embodiments said fibrotic condition may comprise one or more of glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis. In some embodiments, said fibrotic condition may comprise one or more of GSD III, GSD IX, Non Alcoholic Steatohepatitis, cirrhosis of the liver and/or pancreas, scleroderma, idiopathic pulmonary fibrosis, psoriasis, alcoholic fatty liver disease, Dupuytren's disease, and/or any combination thereof.

According to the methods and compositions of the present disclosure, the crystal forms of Compound 1 and Compound 1-A described herein may be administered to a subject for the treatment, amelioration, prevention, or cure of a fibrotic condition, or a condition for which fibrosis is a symptom or sequela. According to the methods and composition as disclosed herein, said fibrotic condition or condition having fibrosis as a sequela may further comprise chronic inflammation. According to the methods and compositions as disclosed herein, said fibrotic condition or condition having fibrosis as a sequela may further comprise activation of one or more TGF-β dependent signaling pathways. According to the methods and compositions as disclosed herein, said fibrotic condition or condition having fibrosis as a sequela may further comprise activation and/or repression of one or more Thyroid Receptor Beta (TRβ) dependent signaling pathways. According to the methods and compositions as disclosed herein, said fibrotic condition or condition having fibrosis as a sequela may further comprise the involvement of signaling pathways responsive to triiodothyronine (T3), thyroxine (T4), any combination thereof, or mimetics thereof. According to the methods and compositions as disclosed herein, said fibrotic condition or condition having fibrosis as a sequela may further comprise the involvement of receptors responsive to T3, T4, any combination thereof, or mimetics thereof. In some embodiments according to the methods and compositions disclosed herein, said fibrotic condition or condition having fibrosis as a sequela may comprise the involvement of TRβ. In some embodiments according to the methods and compositions disclosed herein, said fibrotic condition or condition having fibrosis as a sequela may comprise one or more conditions which are prevented, ameliorated, or cured by the administration of one or more agonists of TRB. In some embodiments according to the methods and compositions disclosed herein, said fibrotic condition or condition having fibrosis as a sequela may comprise one or more conditions which are prevented, ameliorated, or cured by the administration of one or more of compounds described herein. In some embodiments, said compounds disclosed herein may be co-administered with one or more excipients. In some embodiments, said compounds disclosed herein may be administered prior to, during, or after a surgical intervention, phototherapy, or ultrasound therapy.

In some embodiments, the compositions and methods described herein provide compositions and methods for the treatment, amelioration, prevention or cure of collagen deposition. In some embodiments, said collagen deposition comprises and abnormal or excessive deposition of collagen. In some embodiments, said collagen deposition may comprise abnormal or excessive deposition of Type I collagen. In some embodiments, said collagen deposition may comprise abnormal or excessive deposition of Collagen Type Ia. In some embodiments, said collagen deposition may comprise abnormal or excessive deposition of Type III collagen. According to the methods and compositions as disclosed herein, said collagen deposition may further comprise the involvement of receptors responsive to T3, T4, any combination thereof, or mimetics thereof. In some embodiments according to the methods and compositions disclosed herein, said collagen deposition may comprise the involvement of TRβ. In some embodiments according to the methods and compositions disclosed herein, said collagen deposition may be prevented, ameliorated, or cured by the administration of one or more agonists of TRI. In some embodiments according to the methods and compositions disclosed herein, said collagen deposition may be prevented, ameliorated, or cured by the administration of one or more crystalline forms of Compound 1 and/or Compound 1-A. In some embodiments, said one or more crystalline forms of Compound 1 and Compound 1-A may be coadministered with one or more excipients. In some embodiments, one or more crystalline forms of Compound 1 and/or Compound 1-A may be administered prior to, during, or after a surgical intervention, phototherapy, or ultrasound therapy.

In some embodiments, administration of Compound 1 and/or Compound 1-A, and crystal forms thereof described herein or compositions comprising Compound 1 and/or Compound 1-A, and crystal forms thereof as disclosed herein results in a reduction in the expression of the Cola1, Col3a1, αSMA, and/or Galectin1 genes or any combination or product thereof in the subject to which said combination is administered. In some embodiments, administration of one or more crystalline forms of Compound 1 and/or Compound 1-A results in a reduction in the degree of fibrosis observable by histology, histochemistry, immunohistochemistry, or the like, and/or reduction s in the amount, accumulation, or distribution of type 1 collagen and/or hydroxyproline or any combination thereof in the subject to which said combination is administered. In some embodiments, administration of one or more crystalline forms of Compound 1 and/or Compound 1-A results in a reduction in total serum lipids, total serum cholesterol, total serum triglycerides, total liver lipids, total liver cholesterol, total liver triglycerides, or any combination thereof.

### EXAMPLES

Additional embodiments are disclosed in further detail in the following examples, which are not in any way intended to limit the scope of the claims.

### X-Ray Powder Diffraction (XRPD)

The Rigaku Smart-Lab X-ray diffraction system was configured for reflection Bragg-Brentano geometry using a line source X-ray beam. The x-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 ° 2θ using a continuous scan of 6 °2θ per minute with an effective step size of 0.02 °2θ.

### Thermogravimetric Analysis (TG)

Thermogravimetric analyses was carried out using a TA Instruments Q50 instrument. The instrument balance was calibrated using class M weights and the temperature calibration was performed using alumel. The nitrogen purge was ˜40 mL per minute at the balance and ˜60 mL per minute at the furnace. Each sample was placed into a pre-tared platinum pan and heated from 20° C. to 350° C. at a rate of 10° C. per minute.

### Differential Scanning Calorimetry Analysis (DSC)

Differential scanning calorimetry (DSC) was analyses were carried out using a TA Instruments Q2000 instrument. The instrument temperature calibration was performed using indium. The DSC cell was kept under a nitrogen purge of ˜50 mL per minute during each analysis. The sample was placed in a standard, crimped, aluminum pan and was heated from 25° C. to 350° C. at a rate of 10° C. per minute.

### ¹H Nuclear Magnetic Resonance (NMR)

The ¹H-NMR spectra were acquired on a Bruker Avance U 400 spectrometer. Samples were prepared by dissolving material in DMSO-d₆. The solutions were filtered and placed into individual 5-mm NMR tubes for subsequent spectral acquisition. The temperature controlled (295 K) ¹H-NMR spectra acquired on the Avance U 400 utilized a 5-mm cryoprobe operating at an observing frequency of 400.18 MHz.

### Karl Fischer (KF) Analyses

Karl Fischer analyses were carried out using a Mettler-Toledo C20 Coulometric KF titrator. The instrument was calibrated using a Hydranal water standard containing 1% water. The titrant was a Hydranal methanol solution. The sample was analyzed in duplicate.

### Dynamic Vapor Sorption (DVS)

DVS analysis was carried out using a TA Instruments Q5000 Dynamic Vapor Sorption analyzer. The instrument was calibrated with standard weights and a sodium bromide standard for humidity. Approximately 10-25 mg of sample was loaded into a metalcoated quartz pan for analysis. The sample was analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion was not met, after one hour. The percent weight change values were calculated using Microsoft Excel®.

### Example 1: Polymorph Screen Experiments the Compound 1

Polymorph screen experiments for Compound 1 were conducted with various solvents under various conditions. One of the following crystallization methods were used in order to prepare polymorphic forms of Compound 1.

Cooling Experiments: Approximately 20 mg of Compound 1 was dissolved in minimal solvent at a specified temperature. In cases where solubility was very high, antisolvent was used to prevent the use of too much material. The solution was then allowed to cool to room temperature. If no solids were observed within 1 day, the sample was moved to the refrigerator (2-8° C.). If no solids were observed after being in the refrigerator for 1-2 days, the sample was moved to the freezer (−25 to −10° C.). If no solid was observed after being in the freezer for several days, the sample was removed and allowed to evaporate at ambient temperature.

Evaporation Experiments: Approximately 20 mg of Compound 1 was dissolved in minimal solvent at room temperature in a glass vial. The vial was left uncapped in a fume hood at ambient temperature and left to evaporate.

Milling Experiments: Approximately 20 mg of Compound 1 was placed in a PEEK grinding cup with stainless steel ball. The volume in grinding cup was approximately 2 mL. In some cases, solvent (10 μL) was added. The cup was placed on a Retsch mill at 100% power for 20 minutes. The resulting solids were analyzed by XRPD.

Flash Precipitation: Approximately 20 mg of Compound 1 was dissolved in minimal solvent at a specified temperature in a glass vial. Approximately 5-10 volumes of cold antisolvent (from either refrigerator or freezer) was then added to the vial. If no solids formed, sample was placed in freezer (−25 to −10° C.) for several days. If no solid formed in freezer, the sample was removed and allowed to evaporate at ambient.

Antisolvent Precipitation: Approximately 20 mg Compound 1 was dissolved in minimal solvent at a specified temperature. Antisolvent was then added slowly until a turbid solution formed. The solution was allowed to stir overnight. The next day, if no solids formed, additional antisolvent was added. This was repeated daily until either solid precipitated or approximately 5-10 volumes of antisolvent had been added. If no solids formed, the sample was placed in the freezer (−25 to −10° C.) for several days. If no solid formed in freezer, the sample was removed and allowed to evaporate at ambient.

Slurry: Approximately 20 mg of Compound 1 was placed in a vial and solvent added such that not all of the solid dissolved. The slurry was then stirred magnetically at a specified temperature. The solid was then recovered by vacuum filtration.

The results of the polymorph screen experiments are provided in Table 1.

TABLE 1
Polymorph Screens for Compound 1

| | | | XRPD |
|---|---|---|---|
| Method | Solvent^{a} | Conditions^{b} | Pattern^{c} |
| cooling | acetone | hex AS, 50° C. to −15° C.; E | D |
| | ACN | 50° C. to −15° C.; E | A |
| | 1,4-dioxane | 50° C. to −1 5° C.; E | D + NC |
| | DMF | tol AS, 50° C. to −15° C.; E | F + NC |
| | EtOH | hex AS, 50° C. to −15° C.; E | A + D |
| | EtOAc | hex AS, 50° C. to −15° C.; E | A |
| | Et₂O | 50° C. to −15° C. | A + pks |
| | MeOH | tol AS, 50° C. to −15° C.; E | A + C + pks |
| | MEK | hex AS, 50° C. to −15° C. | A + D |
| | 2-MeTHF | hex AS, 50° C. to −15° C.; E | D + A |
| | 2-PrOH | hex AS, 50° C. to −15° C.; E | D + A |
| | | | (min) |
| | THF | hex AS, 50° C. to −15° C. | D |
| | acetone/water (95/5) | hex AS, 50° C. to −15° C.; E | disorder |
| | ACN/water (95/5) | tol AS, 50° C. to −15° C. | E + NC |
| | EtOH/water (95/5) | 50° C. to −15° C.; E | D |
| | MeOH/water (95/5) | tol AS, 50° C. to −15° C.; E | C (LC) |
| | 2-PrOH/water (95/5) | 50° C. to −15° C.; E | B + E |
| | THF/water (95/5) | hex AS, 50° C. to −15° C.; E | D + NC |
| evaporation | acetone | open vial, RT | C (LC) |
| | ACN | open vial, RT | C (LC) |
| | 1,4-dioxane | open vial, RT | C (LC) |
| | DMF | open vial, RT | F |
| | EtOH | open vial, RT | C (LC) |
| | EtOAc | open vial, RT | A + C + |
| | | | 3.5 pk |
| | Et₂O | open vial, RT | A + C + |
| | | | 3.5 pk |
| | MeOH | open vial, RT | C (LC) |
| | MEK | open vial, RT | A + C + |
| | | | 3.5 pk |
| | 2-MeTHF | open vial, RT | A + C |
| | 2-PrOH | open vial, RT | C |
| | THF | open vial, RT | NC + C |
| | acetone/water (95/5) | open vial, RT | C (LC) |
| | ACN/water (95/5) | open vial, RT | C (LC) |
| | EtOH/water (95/5) | open vial, RT | NC + C |
| | MeOH/water (95/5) | open vial, RT | NC + C |
| | 2-PrOH/water (95/5) | open vial, RT | C |
| | THF/water (95/5) | open vial, RT | NC + pks |
| mill | acetone | RT, ~20 mins | A |
| | ACN | RT, ~20 mins | A |
| | EtOH | RT, ~20 mins | A |
| | EtOAc | RT, ~20 mins | A |
| | MeOH | RT, ~20 mins | A |
| | MEK | RT, ~20 mins | A |
| | 2-PrOH | RT, ~20 mins | B + NC |
| | THF | RT, ~20 mins | A |
| | water | RT, ~20 mins | A |
| | none | RT, ~20 mins | A (LC) |
| flash | acetone | hex AS, 60° C. to −15° C. | A |
| precipitation | | water AS, 60° C. to 5° C.; E | disorder |
| | ACN | tol AS, 60° C. to −15° C.; E | A |
| | dioxane | hex AS, 60° C. to −15° C. | A + NC |
| | DMF | tol AS, 60° C. to −15° C. | NC + pk |
| | EtOH | hex AS, 60° C. to −15° C.; E | E + NC |
| | | water AS, 60° C. to 5° C. | C + pks |
| | EtOAc | hex AS, 60° C. to −15° C. | A |
| | MeOH | tol AS, 60° C. to −15° C. | E + pks |
| | | water AS, 60° C. to 5° C.; E | disorder |
| | MEK | hex AS, 60° C. to −15° C. | A |
| | 2-MeTHF | hex AS, 60° C. to −15° C. | A + E |
| | 2-PrOH | hex AS, 60° C. to −15° C.; E | E + NC |
| | | water AS, 60° C. to 5° C.; E | disorder |
| | THF | hex AS, 60° C. to −15° C. | NC + pk |
| | | water AS, 60° C. to 5° C. | D |
| anti-solvent | acetone | DCM AS, RT to −15° C.; E | A + E |
| precipitation | | hex AS, RT | A |
| | | water AS, RT to 5° C. | NC + pks |
| | ACN | DCM AS, RT to −15° C.; E | NC + pks |
| | | tol AS, RT to −15° C.; E | A + D |
| | | water AS, RT to 5° C. | NC + pks |
| | dioxane | DCM AS, RT to −15° C.; E | A + NC |
| | | hex AS, RT to −15° C.; E | NC + pks |
| | | tol AS, RT to −15° C. | A |
| | | water AS, RT to 5° C.; E | disorder |
| anti-solvent | EtOH | hex AS, RT to −15° C. | disorder |
| precipitation | | water AS, RT to 5° C. | NC + pks |
| | EtOAc | hex AS, RT | A |
| | Et₂O | DCM AS, RT to −15° C.; E | E + NC |
| | | hex AS, RT | A |
| | MeOH | DCM AS, RT to −15° C.; E | E + NC |
| | | tol AS, RT to −15° C.; E | disorder |
| | | water AS, RT | C (LC) |
| | MEK | DCM AS, RT to −15° C.; E | E + NC |
| | | hex AS, RT | A |
| | | tol AS, RT to −15° C.; E | A |
| | 2-PrOH | DCM AS, RT to −15° C.; E | disorder |
| | | hex AS, RT to −15° C. | NC + pk |
| | | tol AS, RT to −15° C.; E | disorder |
| | | water AS, RT to 5° C. | disorder |
| | THF | DCM AS, RT to −15° C.; E | D + NC |
| | | hex AS, RT | D |
| | | tol AS, RT to −15° C.; E | NC + pks |
| | | water AS, RT to 5° C. | disorder |
| slurry | DCM | 40° C., 6 days | A |
| | water | 40° C., 6 days | A |
| rotovap | acetone | 50° C. bath; vac. desic. | A |
| | MeOH | 50° C. bath; vac. desic.; hex | A + NC |
| | | AS, air purge | |
| | THF | 50° C. bath; vac. desic.; hex | A + NC |
| | | AS, air purge | |
| heat/humidity | water vapor | RT, 59% RH, 7 days | A |
| | | RT, 75% RH, 7 days | A |
| | | RT, 97% RH, 7 days | A |
| | | 40° C., 75% RH, 7 days | A |
| | none | RT, 0% RH, 7 days | A |
| lyophilization | 1,4-dioxane | LY, dry ice freeze, RT | NC + pks |

^{a}ACN = acetonitrile; AS = anti-solvent; DMF = dimethylformamide; EtOH = ethanol; EtOAc = ethyl acetate; Et₂O = diethyl ether; hex = hexanes; MeOH = methanol; MEK = methyl ethyl ketone; 2-MeTHF = 2-methyl tetrahydrofuran; 2-PrOH = 2-propanol; THF = tetrahydrofuran; tol = toluene
^{b}AS = antisolvent; E = evaporation; RH = relative humidity; RT = room temperature
^{c}LC = low crystallinity; NC = non-crystalline; pk = unidentified peak

### Example 2: Competitive Slurry Experiments for Compound 1

Solutions (˜2 mL) were saturated with Form A of Compound 1. Once saturated, they were centrifuged and the mother liquors were transferred to HPLC vials containing equal amounts (approximately 10 mg each) of Forms A, C, D, and E. The samples were then stirred magnetically at a specified temperature for 3 days. Solids were collected by vacuum filtration. The results of these slurries are presented below in Table 2. Form A was obtained from all of the experiments, indicating that form A is the most stable form under the conditions tested.

TABLE 2
Interconversion Studies for Crystal
Forms of Compound of Formula (I)

| Starting Material | | | XRPD |
|---|---|---|---|
| Crystal Forms | Solvent System | Conditions | Result |
| A, C, D, E | 1:1 | 60° C., 3 days | Form A |
| | ethyl acetate:hexane | | |
| | 2:1 | 25° C., 3 days | Form A |
| | ethyl acetate:hexane | | |
| | 1:1 | 2° C., 3 days | Form A |
| | ethyl acetate:hexane | | |
| | Dry EtOAc | Room temp, 3 days | Form A |
| | water | Room temp, 3 days | Form A |

### Example 3: Polymorph Screen Experiments for Compound 1-A

Polymorph screen experiments for the (2-methylamino)ethanol (“deanol”) salts were carried out by combining Compound 1 and deanol with various solvents under various conditions. Several polymorphs were prepared as indicated below. All of the crystalline salts exhibited a 1:1 stoichiometry of Compound 1:deanol. The results are provided in Table 3 below.

TABLE 3
Polymorph Screens for Compound 1-A

| | | XRPD |
|---|---|---|
| Solvent | Conditions^{a} | Pattern^{b} |
| Acetone | Dissolve 20 mg Compound 1 in 0.5 | Form 1 |
| | mL of acetone. Add solution to vial | |
| | containing 1 molarequivalent of | |
| | deanol. Sonicate; much solid. Stir at | |
| | RT for 3 days. | |
| | Combine 15 mg Compound 1, 1 | Form 3 |
| | molar equivalent deanol, and 1 mL | |
| | acetone. Add 25 μL water, sonicate; | |
| | much solid. Stir at RT for 2 days | |
| Acetonitrile | Dissolve 20 mg Compound 1 in 2.5 | Form 4 |
| (ACN) | mL of ACN. Add solution to vial | |
| | containing 1 molar equivalent of | |
| | deanol. Sonicate; much solid. Stir at | |
| | RT for 3 days. | |
| | Placed in 70° C. oven, 1 day | Form 4 |
| 1,4-Dioxane | Dissolve 20 mg Compound 1 in 0.5 | Form 1 |
| | mL of dioxane. Add solution to vial | |
| | containing 1 molar equivalent of | |
| | deanol. Sonicate; much solid. Stir at | |
| | RT for 3 days. | |
| Ethanol | Place 433 mg of Compound 1 in | Form 3 |
| (EtOH) | vial. Add 1 molar equivalent of | |
| | deanol pre-dissolved in 3 mL of | |
| | EtOH. Sonicated; clear. Let | |
| | partially evaporate until solids form, | |
| | then stir at RT overnight. Recover | |
| | solids by filtration | |
| | Dissolve 20 mg Compound 1 in 0.5 | Form 3 |
| | mL of EtOH. Add solution to vial | |
| | containing 1 molar equivalent of | |
| | deanol. Sonicate; much solid. Stir at | |
| | RT; no solid after 2 hours. | |
| | Uncapped, allowed to evaporate at | |
| | RT | |
| | Dissolve 20 mg Compound 1 in 0.5 | Form 3 |
| | mL of EtOH. Add solution to vial | |
| | containing 1 molar equivalent of | |
| | deanol. Sonicate; clear solution. | |
| | Add about 6 mL dichloromethane, | |
| | clear, put in freezer. Solids after 1 | |
| | day | |
| Ethanol | Dissolve 15 mg Compound 1 and 1 | Form 3 |
| (EtOH) | equ deanol in 0.25 mL EtOH at 60° | |
| | C. Cooled from RT to 2° C.; clear | |
| | film. Let evaporate at RT; clear | |
| | film. Placed in 40° C./75% RH | |
| | chamber, 1 day | |
| | Dissolve 15 mg Compound 1 and 1 | Form 3 |
| | equ deanol in 0.75 mL EtOH. Add | |
| | IPrOAc dropwise; becomes turbid | |
| | after 2 mL. Placed in −15° C. | |
| | Solids after 1 day | |
| | Dissolve 36 mg Compound 1 and 1 | Form 1 |
| | equ deanol in 0.6 mL EtOH at 60° | |
| | C. Add hexanes dropwise; turbid | |
| | after 1/2 mL. Let cool to RT, much | |
| | solid. Stir at RT for 2 days | |
| Ethyl Acetate | Dissolve 20 mg Compound 1 in 0.5 | Form 4 (LC) |
| (EtOAc) | mL of EtOAc. Add solution to vial | |
| | containing 1 molar equivalent of | |
| | deanol. Sonicate; much solid. Stir | |
| | at RT for 3 days. | |
| | Placed in 70° C. oven, 1 day | Form 4 |
| Ethyl Ether | Dissolve 20 mg Compound 1 in 1 | Form 1 + |
| (Et₂O) | mL of Et₂O. Add solution to vial | NC |
| | containing 1 molar equivalent of | |
| | deanol. Sonicate; much solid. Stir | |
| | at RT for 3 days. | |
| Isopropyl | Dissolve 20 mg Compound 1 in | Form 3 |
| Acetate | 0.5 mL of IPrOAc. Add solution | |
| (IPrOAc) | to vial containing 1 molar | |
| | equivalent of deanol. Sonicate; | |
| | much solid. Stir at RT for 3 days. | |
| Methanol | Dissolve 20 mg Compound 1 in 0.5 | Form 1 (LC) |
| (MeOH) | mL of MeOH. Add solution to vial | |
| | containing 1 molar equivalent of | |
| | deanol. Sonicate; clear solution. | |
| | Add Et₂O until turbid, about 3 mL. | |
| | Stir at RT 3 days. | |
| | Dissolve 20 mg Compound 1 in 0.5 | — |
| | mL of MeOH. Add solution to vial | |
| | containing 1 molar equivalent of | |
| | deanol. Sonicate; clear solution. | |
| | Let evaporate; clear glass. Placed | |
| | in 70° C. oven, one day; | |
| | unchanged. Glass broken up with | |
| | spatula, returned to 70° C. oven, 1 | |
| | day; clear glass | |
| | Dissolve 15 mg Compound 1 and 1 | Form 3 + |
| | equ deanol in 0.25 mL MeOH. Add | NC |
| | toluene dropwise until turbid; | |
| | 2 mL. Cooled from RT to −15° C. | |
| | Solids after 1 day | |
| Methyl Ethyl | Dissolve 20 mg Compound 1 in 0.5 | Form 1 (LC) |
| Ketone | mL of MEK. Add solution to vial | |
| (MEK) | containing 1 molar equivalent of | |
| | deanol. Sonicate; much solid. Stir | |
| | at RT for 3 days. | |
| 1-Propanol | Dissolve 20 mg Compound 1 in 0.5 | Form 3 |
| (1-PrOH) | mL of 1-PrOH. Add solution to | |
| | vial containing 1 molar equivalent | |
| | of deanol. Sonicate; much solid. | |
| | Stir at RT for 3 days. | |
| 2-Propanol | Dissolve 20 mg Compound 1 in 0.5 | Form 1 |
| (2-PrOH) | mL of 2-PrOH. Add solution to | |
| | vial containing 1 molar equivalent | |
| | of deanol. Sonicate; much solid. | |
| | Stir at RT for 3 days. | |
| Tetrahydrofuran | Dissolve 20 mg Compound 1 in | Form 1 |
| (THF) | 0.5 mL of THF. Add solution to | |
| | vial containing 1 molar equivalent | |
| | of deanol. Sonicate; much solid. | |
| | Stir at RT for 3 days. | |
| Water | Add 20 mg Compound 1 to a vial | Form 3 |
| | containing 1 molar equivalent of | |
| | deanol dissolved in 1 mL of water; | |
| | dissolves. Allowed to evaporate at | |
| | RT. | |

^{a}RH = relative humidity; RT = room temperature; SP = sonic probe
^{b}LC = low crystallinity; NC = non-crystalline; pk = peak

### Example 4: Polymorph Screen Experiments for Compound 1-A

Compound 1-A Form 3 was mixed with various solvents under various conditions. Three polymorphs were identified as Forms 1, 2, and 3. All of the crystalline salts exhibited a 1:1 stoichiometry of Compound 1:deanol.

TABLE 4
Polymorph Screens for the Compound of Formula (I)

| Solvent | Conditions^{a} | XRPD Pattern^{b} |
|---|---|---|
| acetone/MeOH | SL, RT, 1 day; 55° C., 2 days | Form 1 (LC) |
| ACN/MeOH | SL, RT, 1 day; 55° C., 2 days | Form 1 (LC) |
| DCM/MeOH | SL, RT, 1 day; 8° C., 2 days | Form 3 |
| Dioxane | C, 65° C. to −15° C.; no solid. E, RT | Form 3 |
| | | (single crystals) |
| EtOH | E, RT | Form 3 (PO) |
| | | (single crystals) |
| EtOAc/MeOH | SL, RT, 1 day; 55° C., 2 days | Form 1 (LC) |
| MeOH | SE, RT (oil); air-dried (glassy) | NC |
| 2-propanol | C, 65° C. to −15° C.; no solid. E, RT | Form 3 |
| | | (single crystals) |
| THF | C, 65° C. to −15° C.; no solid E, RT | Form 1 + |
| | | min Form 3 |
| toluene/MeOH | SL, RT, 1 day; 8° C., 2 days | Form 3 |
| n/a | 20 mg of form 3 heated on TG to 120° C. | Form 3 + pks |
| | and held for 3 minutes. Resulting solid | |
| | analyzed by XRPD | |
| | Sample left at RT, 12 hours (30% RH) | Form 3 |

^{a}C = cool; E = evaporate; RH = relative humidity; RT = room temperature; SL = slurry
^{b}LC = low crystallinity; NC = non-crystalline; pk = peak, PO = preferred orientation

### Example 5: Preparation of Form 3 of Compound 1-A

To a 500 mL Erlenmeyer flask was added Compound 1 (16.089 g) and 40 mL acetone (containing 2.5% water). The mixture was allowed to magnetically stir at room temperature until the solids dissolved. In a separate flask, 2-(dimethylamino)ethanol (“deanol”; 1.05 equivalents, 3.650 g) was dissolved in 10 mL of acetone (containing 2.5% water). The deanol solution was added to the Compound 1 solution over a 10 minute period. Solids began to precipitate after about 2 mL had been added and a thick slurry was formed. An additional 150 mL acetone (containing 2.5% water) was added to the Compound 1 slurry bringing total amount of solvent in the flask to 200 mL. The slurry was stirred magnetically at room temperature overnight. The slurry was then vacuum filtered through Whatman filter paper. The recovered white solid was placed on weighing paper and broken up with a spatula and allowed to air dry for two days. Compound 1 deanol salt (i.e., Compound 1-A) (18.817 g, 93% yield) was recovered.

### Example 6: Characterization of Crystalline Samples

The crystalline solid forms were characterized by a combinations of XRPD, thermogravimetry (TG), DSC and/or solution NMR.

### Crystalline Form A of Compound 1

The XRPD results of crystalline Form A (FIG. 1) show good crystallinity. A melting temperature at approximately 161° C. was observed using differential scanning calorimetry (FIG. 2). Crystalline Form A was moderately hygroscopic dynamic vapor sorption/desorption analysis (FIG. 3).

XRPD measurements of crystalline Form A of Compound 1 were measured. Observed peaks are shown in Table 5. Prominent peaks are listed in Table 6.

TABLE 5
Observed Peaks for Crystalline Form A

| Diffraction | | |
|---|---|---|
| angle °2θ (deg) | d space (Å) | Intensity (%) |
| 5.1 | 17.4 | 93.4 |
| 7.4 | 11.9 | 10.6 |
| 10.1 | 8.8 | 4.2 |
| 12.1 | 7.3 | 7.5 |
| 12.9 | 6.8 | 3.1 |
| 14.7 | 6.0 | 0.6 |
| 16.4 | 5.4 | 100.0 |
| 16.5 | 5.4 | 12.6 |
| 17.3 | 5.1 | 18.2 |
| 18.5 | 4.8 | 7.8 |
| 19.2 | 4.6 | 2.3 |
| 19.5 | 4.5 | 3.4 |
| 20.0 | 4.4 | 0.4 |
| 20.5 | 4.3 | 5.0 |
| 21.0 | 4.2 | 5.1 |
| 21.4 | 4.2 | 9.0 |
| 21.7 | 4.1 | 16.5 |
| 22.1 | 4.0 | 10.4 |
| 22.3 | 4.0 | 1.7 |
| 22.6 | 3.9 | 2.5 |
| 23.8 | 3.7 | 6.9 |
| 24.1 | 3.7 | 19.6 |
| 25.5 | 3.5 | 17.5 |
| 25.9 | 3.4 | 9.6 |
| 26.4 | 3.4 | 0.4 |
| 26.6 | 3.3 | 8.6 |
| 27.7 | 3.2 | 8.3 |
| 28.4 | 3.1 | 0.4 |
| 29.2 | 3.1 | 1.3 |
| 29.8 | 3.0 | 0.6 |

TABLE 6
Prominent Peaks for Crystalline Form A

| Diffraction | | |
|---|---|---|
| angle °2θ (deg) | d space (Å) | Intensity (%) |
| 5.1 | 17.4 | 93.4 |
| 7.4 | 11.9 | 10.6 |
| 10.1 | 8.8 | 4.2 |
| 12.1 | 7.3 | 7.5 |
| 16.4 | 5.4 | 100.0 |
| 17.3 | 5.1 | 18.2 |
| 18.5 | 4.8 | 7.8 |
| 20.5 | 4.3 | 5.0 |
| 21.0 | 4.2 | 5.1 |
| 21.4 | 4.2 | 9.0 |
| 21.7 | 4.1 | 16.5 |
| 22.1 | 4.0 | 10.4 |
| 23.8 | 3.7 | 6.9 |
| 24.1 | 3.7 | 19.6 |
| 25.5 | 3.5 | 17.5 |
| 25.9 | 3.4 | 9.6 |

### Crystalline Form B of Compound 1

The XRPD results of crystalline Form B (FIG. 4) shows poor crystallinity. Crystalline Form B was obtained only during the IPA grind experiment. Crystalline form B was determined to be a solvate of isopropyl alcohol.

XRPD measurements of crystalline Form B of Compound 1 were measured. Observed peaks are shown in Table 7. Prominent peaks are listed in Table 8.

TABLE 7
Observed Peaks for Crystalline Form B

| Diffraction | d space | |
|---|---|---|
| angle °2θ (deg) | (Å) | Intensity (%) |
| 4.0 | 21.8 | 12.6 |
| 7.5 | 11.8 | 45.7 |
| 7.9 | 11.2 | 9.0 |
| 8.1 | 10.9 | 47.9 |
| 10.0 | 8.8 | 32.8 |
| 13.2 | 6.7 | 6.3 |
| 15.4 | 5.7 | 23.6 |
| 15.8 | 5.6 | 6.2 |
| 16.7 | 5.3 | 100.0 |
| 17.6 | 5.0 | 4.1 |
| 18.3 | 4.8 | 28.3 |
| 18.8 | 4.7 | 11.0 |
| 19.3 | 4.6 | 1.9 |
| 20.2 | 4.4 | 24.8 |
| 20.4 | 4.4 | 7.4 |
| 21.0 | 4.2 | 21.9 |
| 21.3 | 4.2 | 7.2 |
| 21.8 | 4.1 | 5.6 |
| 22.3 | 4.0 | 23.0 |
| 22.6 | 3.9 | 10.1 |
| 23.2 | 3.8 | 8.7 |
| 24.1 | 3.7 | 46.8 |
| 25.1 | 3.5 | 27.1 |
| 26.4 | 3.4 | 69.7 |
| 27.6 | 3.2 | 6.9 |
| 28.7 | 3.1 | 32.6 |
| 29.3 | 3.0 | 1.8 |

TABLE 8
Prominent Peaks for Crystalline Form B

| Diffraction | d space | |
|---|---|---|
| angle °2θ (deg) | (Å) | Intensity (%) |
| 7.5 | 11.8 | 45.7 |
| 8.1 | 10.9 | 47.9 |
| 10.0 | 8.8 | 32.8 |
| 15.4 | 5.7 | 23.6 |
| 16.7 | 5.3 | 100.0 |
| 18.3 | 4.8 | 28.3 |
| 18.8 | 4.7 | 11.0 |
| 20.2 | 4.4 | 24.8 |
| 21.0 | 4.2 | 21.9 |
| 22.3 | 4.0 | 23.0 |
| 22.6 | 3.9 | 10.1 |
| 24.1 | 3.7 | 46.8 |
| 25.1 | 3.5 | 27.1 |
| 26.4 | 3.4 | 69.7 |

### Crystalline Form C of Compound 1

The XRPD results of crystalline Form C (FIG. 5) show poor crystallinity. An endotherm at approximately 158° C. was observed using differential scanning calorimetry (FIG. 6), likely indicating the melting point of the crystal. Crystalline Form C became non-crystalline upon heating. Crystalline Form C exhibited a 4.4% weight loss when carried out from 25° C. to 110° C. as measured by thermogravimetric analysis. However, the weight loss was found not to be attributed to solvent.

XRPD measurements of crystalline Form C of Compound 1 were measured. Observed peaks are shown in Table 9. Prominent peaks are listed in Table 10

TABLE 9
Observed Peaks for Crystalline Form C

| Diffraction | | Intensity |
|---|---|---|
| angle °2θ (deg) | d space (Å) | (%) |
| 4.0 | 21.9 | 100.0 |
| 8.0 | 11.0 | 18.9 |
| 11.0 | 8.1 | 57.5 |
| 11.1 | 8.0 | 4.0 |
| 12.0 | 7.4 | 26.9 |
| 13.6 | 6.5 | 5.5 |
| 14.4 | 6.2 | 83.8 |
| 14.6 | 6.0 | 67.2 |
| 15.2 | 5.8 | 7.6 |
| 17.1 | 5.2 | 31.4 |
| 18.5 | 4.8 | 87.3 |
| 19.2 | 4.6 | 18.1 |
| 21.2 | 4.2 | 17.0 |
| 21.7 | 4.1 | 13.6 |
| 24.9 | 3.6 | 11.6 |
| 25.7 | 3.5 | 54.1 |

TABLE 10
Prominent Peaks for Crystalline Form C

| Diffraction | | Intensity |
|---|---|---|
| angle °2θ (deg) | d space (Å) | (%) |
| 4.0 | 21.9 | 100.0 |
| 8.0 | 11.0 | 18.9 |
| 11.0 | 8.1 | 57.5 |
| 12.0 | 7.4 | 26.9 |
| 14.4 | 6.2 | 83.8 |
| 14.6 | 6.0 | 67.2 |
| 17.1 | 5.2 | 31.4 |
| 18.5 | 4.8 | 87.3 |
| 19.2 | 4.6 | 18.1 |
| 21.2 | 4.2 | 17.0 |
| 21.7 | 4.1 | 13.6 |
| 24.9 | 3.6 | 11.6 |
| 25.7 | 3.5 | 54.1 |

### Crystalline Form D of Compound 1

The XRPD results of crystalline Form D (FIG. 7) show good crystallinity. A melting temperature at approximately 166° C. was observed using differential scanning calorimetry (FIG. 8). Thermogravimetric gravimetric analysis showed a 3.875% weight loss when carried out from 25° C. to 140° C., indicating a possible hydrate.

XRPD measurements of crystalline Form D of Compound 1 were measured. Observed peaks are shown in Table 11. Prominent peaks are listed in Table 12.

TABLE 11
Observed Peaks for Crystalline Form D

| Diffraction | | Intensity |
|---|---|---|
| angle °2θ (deg) | d space (Å) | (%) |
| 3.4 | 26.2 | 9.1 |
| 6.1 | 14.4 | 28.4 |
| 7.4 | 12.0 | 9.9 |
| 7.6 | 11.7 | 31.9 |
| 11.3 | 7.9 | 28.3 |
| 11.5 | 7.7 | 6.6 |
| 12.2 | 7.2 | 46.9 |
| 14.7 | 6.0 | 4.4 |
| 16.5 | 5.4 | 13.1 |
| 17.0 | 5.2 | 20.4 |
| 17.3 | 5.1 | 10.9 |
| 17.6 | 5.0 | 5.1 |
| 18.4 | 4.8 | 58.0 |
| 18.6 | 4.8 | 35.6 |
| 19.5 | 4.5 | 32.2 |
| 19.7 | 4.5 | 65.5 |
| 20.7 | 4.3 | 42.1 |
| 21.0 | 4.2 | 4.2 |
| 21.2 | 4.2 | 100.0 |
| 21.7 | 4.1 | 8.1 |
| 22.5 | 3.9 | 19.6 |
| 23.0 | 3.9 | 5.2 |
| 23.3 | 3.8 | 7.8 |
| 24.1 | 3.7 | 1.1 |
| 24.6 | 3.6 | 1.8 |
| 25.2 | 3.5 | 4.6 |
| 25.4 | 3.5 | 5.6 |
| 26.0 | 3.4 | 24.8 |
| 26.3 | 3.4 | 11.9 |
| 26.5 | 3.4 | 3.3 |
| 26.8 | 3.3 | 10.2 |
| 27.8 | 3.2 | 2.2 |
| 28.3 | 3.2 | 7.0 |
| 28.5 | 3.1 | 3.7 |
| 28.8 | 3.1 | 1.4 |
| 29.3 | 3.0 | 7.4 |
| 29.8 | 3.0 | 3.0 |

TABLE 12
Prominent Peaks for Crystalline Form D

| Diffraction angle | | Intensity |
|---|---|---|
| °2θ (deg) | d space (Å) | (%) |
| 6.1 | 14.4 | 28.4 |
| 7.6 | 11.7 | 31.9 |
| 11.3 | 7.9 | 28.3 |
| 12.2 | 7.2 | 46.9 |
| 16.5 | 5.4 | 13.1 |
| 17.0 | 5.2 | 20.4 |
| 17.3 | 5.1 | 10.9 |
| 18.4 | 4.8 | 58.0 |
| 18.6 | 4.8 | 35.6 |
| 19.5 | 4.5 | 32.2 |
| 19.7 | 4.5 | 65.5 |
| 20.7 | 4.3 | 42.1 |
| 21.2 | 4.2 | 100.0 |
| 22.5 | 3.9 | 19.6 |
| 26.0 | 3.4 | 24.8 |
| 26.3 | 3.4 | 11.9 |

### Crystalline Form E of Compound 1

The XRPD results of crystalline Form E (FIG. 9) show poor crystallinity. Endotherms were observed via DSC at 96° C. and 166° C. for crystalline Form E (FIG. 10). Crystalline Form E exhibited a 2.5% weight loss when carried out from 25° C. to 125° C., however, the loss of mass is not attributed to solvent.

XRPD measurements of crystalline Form E of Compound 1 were measured. Observed peaks are shown in Table 13. Prominent peaks are listed in Table 14.

TABLE 13
Observed Peaks for Crystalline Form E

| Diffraction angle | | Intensity |
|---|---|---|
| °2θ (deg) | d space (Å) | (%) |
| 3.3 | 26.7 | 73.4 |
| 5.0 | 17.5 | 100.0 |
| 7.5 | 11.9 | 36.9 |
| 10.0 | 8.9 | 22.7 |
| 12.3 | 7.2 | 18.9 |
| 13.5 | 6.6 | 16.7 |
| 14.9 | 5.9 | 23.5 |
| 16.3 | 5.4 | 69.6 |
| 17.2 | 5.2 | 24.3 |
| 18.3 | 4.9 | 13.0 |
| 19.4 | 4.6 | 49.5 |
| 20.4 | 4.4 | 16.1 |
| 21.3 | 4.2 | 20.3 |
| 21.7 | 4.1 | 43.1 |
| 23.8 | 3.7 | 10.1 |
| 24.1 | 3.7 | 20.6 |
| 24.7 | 3.6 | 41.6 |
| 25.5 | 3.5 | 27.6 |
| 26.6 | 3.4 | 52.8 |
| 27.7 | 3.2 | 9.3 |

TABLE 14
Prominent Peaks for Crystalline Form E

| Diffraction angle | | Intensity |
|---|---|---|
| °2θ (deg) | d space (Å) | (%) |
| 3.3 | 26.7 | 73.4 |
| 5.0 | 17.5 | 100.0 |
| 7.5 | 11.9 | 36.9 |
| 14.9 | 5.9 | 23.5 |
| 16.3 | 5.4 | 69.6 |
| 17.2 | 5.2 | 24.3 |
| 19.4 | 4.6 | 49.5 |
| 21.7 | 4.1 | 43.1 |
| 24.7 | 3.6 | 41.6 |
| 25.5 | 3.5 | 27.6 |
| 26.6 | 3.4 | 52.8 |

### Crystalline Form F of Compound 1

The XRPD results of crystalline Form F (FIG. 11). Crystalline Form B was obtained only during a DMF evaporation experiment and determined to be a dimethylformamide solvate. DSC results indicate an endotherm at 96° C. for crystalline Form F (FIG. 12).

XRPD measurements of crystalline Form F of Compound 1 were measured. Observed peaks are shown in Table 15. Prominent peaks are listed in Table 6.

TABLE 15
Observed Peaks for Crystalline Form F

| Diffraction angle | | Intensity |
|---|---|---|
| °2θ (deg) | d space (Å) | (%) |
| 8.3 | 10.6 | 15.5 |
| 12.7 | 7.0 | 8.0 |
| 13.8 | 6.4 | 8.1 |
| 15.5 | 5.7 | 25.4 |
| 15.6 | 5.7 | 17.2 |
| 15.9 | 5.6 | 39.3 |
| 16.7 | 5.3 | 13.4 |
| 17.0 | 5.2 | 49.2 |
| 17.3 | 5.1 | 27.3 |
| 17.9 | 4.9 | 21.5 |
| 18.6 | 4.8 | 100.0 |
| 20.8 | 4.3 | 49.4 |
| 22.0 | 4.0 | 33.1 |
| 23.0 | 3.9 | 40.4 |
| 23.6 | 3.8 | 35.6 |
| 23.8 | 3.7 | 34.1 |
| 24.1 | 3.7 | 5.1 |
| 25.5 | 3.5 | 32.0 |
| 25.7 | 3.5 | 51.5 |
| 26.0 | 3.4 | 59.4 |
| 26.7 | 3.3 | 26.3 |
| 29.1 | 3.1 | 6.8 |
| 29.7 | 3.0 | 9.5 |

TABLE 16
Prominent Peaks for Crystalline Form F

| Diffraction angle | | Intensity |
|---|---|---|
| °2θ (deg) | d space (Å) | (%) |
| 8.3 | 10.6 | 15.5 |
| 15.5 | 5.7 | 25.4 |
| 15.6 | 5.7 | 17.2 |
| 15.9 | 5.6 | 39.3 |
| 17.0 | 5.2 | 49.2 |
| 17.3 | 5.1 | 27.3 |
| 17.9 | 4.9 | 21.5 |
| 18.6 | 4.8 | 100.0 |
| 20.8 | 4.3 | 49.4 |
| 22.0 | 4.0 | 33.1 |
| 23.0 | 3.9 | 40.4 |
| 23.6 | 3.8 | 35.6 |
| 23.8 | 3.7 | 34.1 |
| 25.5 | 3.5 | 32.0 |
| 25.7 | 3.5 | 51.5 |
| 26.0 | 3.4 | 59.4 |
| 26.7 | 3.3 | 26.3 |

### Crystalline Form 1 of Compound 1-A

The XRPD results of crystalline Form 1 (FIG. 13) show good crystallinity. A melting temperature at approximately 139° C. was observed using differential scanning calorimetry. Crystalline Form A was moderately hygroscopic and had a melting point of about 139° C.

XRPD measurements of crystalline Form 1 of Compound 1-A were measured. Observed peaks are shown in Table 17. Prominent peaks are listed in Table 18.

TABLE 17
Observed Peaks for Crystalline Form 1

| Diffraction angle | | Intensity |
|---|---|---|
| °2θ (deg) | d space (Å) | (%) |
| 6.1 | 14.5 | 91.4 |
| 8.4 | 10.6 | 8.9 |
| 12.0 | 7.4 | 8.2 |
| 12.2 | 7.2 | 12.8 |
| 12.7 | 7.0 | 6.3 |
| 13.3 | 6.7 | 10.6 |
| 13.6 | 6.5 | 18.3 |
| 14.0 | 6.3 | 8.4 |
| 14.4 | 6.1 | 8.6 |
| 14.9 | 5.9 | 2.9 |
| 15.9 | 5.6 | 6.9 |
| 16.5 | 5.4 | 51.8 |
| 17.0 | 5.2 | 8.2 |
| 17.3 | 5.1 | 23.0 |
| 18.3 | 4.9 | 100.0 |
| 19.3 | 4.6 | 7.8 |
| 19.9 | 4.5 | 41.5 |
| 20.2 | 4.4 | 17.1 |
| 20.5 | 4.3 | 35.5 |
| 21.4 | 4.1 | 42.2 |
| 21.9 | 4.1 | 13.7 |
| 22.3 | 4.0 | 9.0 |
| 22.5 | 4.0 | 21.4 |
| 22.6 | 3.9 | 5.6 |
| 22.8 | 3.9 | 17.5 |
| 23.0 | 3.9 | 16.8 |
| 23.4 | 3.8 | 9.0 |
| 24.0 | 3.7 | 8.1 |
| 24.4 | 3.6 | 5.7 |
| 25.6 | 3.5 | 14.3 |
| 26.3 | 3.4 | 8.1 |
| 27.0 | 3.3 | 9.4 |
| 28.1 | 3.2 | 9.8 |
| 28.9 | 3.1 | 4.8 |
| 29.1 | 3.1 | 3.1 |
| 29.4 | 3.0 | 1.9 |
| 29.9 | 3.0 | 4.6 |

TABLE 18
Prominent Peaks for Crystalline Form 1

| Diffraction angle | | Intensity |
|---|---|---|
| °2θ (deg) | d space (Å) | (%) |
| 6.1 | 14.5 | 91.4 |
| 12.2 | 7.2 | 12.8 |
| 13.3 | 6.7 | 10.6 |
| 13.6 | 6.5 | 18.3 |
| 16.5 | 5.4 | 51.8 |
| 17.3 | 5.1 | 23.0 |
| 18.3 | 4.9 | 100.0 |
| 19.9 | 4.5 | 41.5 |
| 20.2 | 4.4 | 17.1 |
| 20.5 | 4.3 | 35.5 |
| 21.4 | 4.1 | 42.2 |
| 21.9 | 4.1 | 13.7 |
| 22.5 | 4.0 | 21.4 |
| 22.8 | 3.9 | 17.5 |
| 23.0 | 3.9 | 16.8 |
| 25.6 | 3.5 | 14.3 |

### Crystalline Form 2 of Compound 1-A

The XRPD results of crystalline Form 2 show crystallinity (FIG. 14). Crystal Form 2 was found to have a melting temperature of about 117° C. and determined to be moderately hygroscopic.

XRPD measurements of crystalline Form 2 of Compound 1-A were measured. Observed peaks are shown in Table 19. Prominent peaks are listed in Table 20.

TABLE 19
Observed Peaks for Crystalline Form 2

| Diffraction angle | | |
|---|---|---|
| °2θ (deg) | d space (Å) | Intensity (%) |
| 5.7 | 15.5 | 38.7 |
| 6.2 | 14.3 | 43.5 |
| 8.0 | 11.0 | 1.0 |
| 8.6 | 10.3 | 1.4 |
| 11.4 | 7.8 | 3.9 |
| 12.3 | 7.2 | 2.5 |
| 12.9 | 6.9 | 4.7 |
| 13.6 | 6.5 | 2.3 |
| 14.1 | 6.3 | 2.0 |
| 15.5 | 5.7 | 5.8 |
| 16.2 | 5.5 | 0.7 |
| 16.4 | 5.4 | 7.7 |
| 17.0 | 5.2 | 23.2 |
| 18.5 | 4.8 | 100.0 |
| 20.2 | 4.4 | 3.9 |
| 20.4 | 4.4 | 4.0 |
| 20.6 | 4.3 | 7.6 |
| 21.2 | 4.2 | 5.5 |
| 22.0 | 4.0 | 17.0 |
| 22.5 | 3.9 | 3.5 |
| 22.8 | 3.9 | 0.6 |
| 23.3 | 3.8 | 4.4 |
| 23.4 | 3.8 | 2.5 |
| 24.0 | 3.7 | 2.2 |
| 24.3 | 3.7 | 1.2 |
| 24.7 | 3.6 | 2.2 |
| 25.5 | 3.5 | 1.0 |
| 26.1 | 3.4 | 4.5 |
| 26.8 | 3.3 | 0.9 |
| 27.5 | 3.2 | 1.4 |
| 27.9 | 3.2 | 1.5 |
| 28.5 | 3.1 | 1.4 |
| 28.9 | 3.1 | 1.3 |
| 29.5 | 3.0 | 2.3 |
| 29.9 | 3.0 | 2.8 |

TABLE 20
Prominent Peaks for Crystalline Form 2

| Diffraction angle | | |
|---|---|---|
| °2θ (deg) | d space (Å) | Intensity (%) |
| 5.7 | 15.5 | 38.7 |
| 6.2 | 14.3 | 43.5 |
| 15.5 | 5.7 | 5.8 |
| 16.4 | 5.4 | 7.7 |
| 17.0 | 5.2 | 23.2 |
| 18.5 | 4.8 | 100.0 |
| 20.6 | 4.3 | 7.6 |
| 21.2 | 4.2 | 5.5 |
| 22.0 | 4.0 | 17.0 |
| 23.3 | 3.8 | 4.4 |
| 26.1 | 3.4 | 4.5 |

### Crystalline Form 3 of Compound 1-A

The XRPD results of crystalline Form 3 (FIG. 15) show good crystallinity. A melting temperature at approximately 156° C. was observed using differential scanning calorimetry. Another endotherm was observed at about 107° C. (FIG. 16). Crystalline Form 3 exhibited a 3.85% weight loss when carried out from 25° C. to 90° C. and a 2.09% weight loss when carried out from 90° C. to 150° C. DVS data (not shown) indicates slight water uptake. Karl Fisher titration indicates that Crystalline Form 3 is a monohydrate.

XRPD measurements of crystalline Form 3 of Compound 1-A were measured. Observed peaks are shown in Table 21. Prominent peaks are listed in Table 22.

TABLE 21
Observed Peaks for Crystalline Form 3

| Diffraction angle | | |
|---|---|---|
| °2θ (deg) | d space (Å) | Intensity (%) |
| 6.0 | 14.7 | 22.7 |
| 8.4 | 10.5 | 6.8 |
| 10.2 | 8.7 | 3.2 |
| 10.5 | 8.4 | 4.5 |
| 11.9 | 7.4 | 5.2 |
| 12.2 | 7.3 | 4.1 |
| 13.3 | 6.7 | 23.7 |
| 14.4 | 6.1 | 12.0 |
| 14.8 | 6.0 | 73.5 |
| 15.6 | 5.7 | 8.0 |
| 16.1 | 5.5 | 68.8 |
| 17.8 | 5.0 | 23.7 |
| 17.9 | 5.0 | 11.6 |
| 18.7 | 4.7 | 46.1 |
| 20.5 | 4.3 | 38.4 |
| 20.5 | 4.3 | 5.4 |
| 20.8 | 4.3 | 25.9 |
| 21.7 | 4.1 | 99.6 |
| 22.4 | 4.0 | 3.0 |
| 22.9 | 3.9 | 20.2 |
| 23.8 | 3.7 | 100.0 |
| 24.3 | 3.7 | 4.2 |
| 24.9 | 3.6 | 10.3 |
| 25.1 | 3.5 | 48.3 |
| 25.8 | 3.4 | 1.6 |
| 26.2 | 3.4 | 7.3 |
| 26.6 | 3.3 | 12.8 |
| 27.3 | 3.3 | 8.5 |
| 27.5 | 3.2 | 1.3 |
| 28.1 | 3.2 | 1.8 |
| 29.8 | 3.0 | 7.1 |

TABLE 22
Prominent Peaks for Crystalline Form 3

| Diffraction angle | | |
|---|---|---|
| °2θ (deg) | d space (Å) | Intensity (%) |
| 6.0 | 14.7 | 22.7 |
| 13.3 | 6.7 | 23.7 |
| 14.4 | 6.1 | 12.0 |
| 14.8 | 6.0 | 73.5 |
| 16.1 | 5.5 | 68.8 |
| 17.8 | 5.0 | 23.7 |
| 17.9 | 5.0 | 11.6 |
| 18.7 | 4.7 | 46.1 |
| 20.5 | 4.3 | 38.4 |
| 20.8 | 4.3 | 25.9 |
| 21.7 | 4.1 | 99.6 |
| 22.9 | 3.9 | 20.2 |
| 23.8 | 3.7 | 100.0 |
| 24.9 | 3.6 | 10.3 |
| 25.1 | 3.5 | 48.3 |
| 26.6 | 3.3 | 12.8 |

### Crystalline Form 4 of Compound 1-A

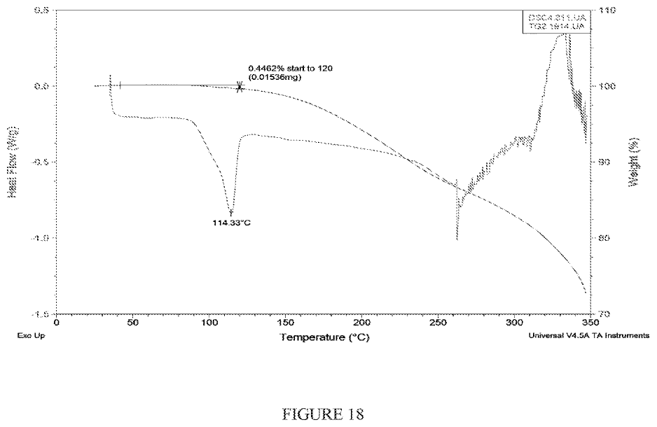

The XRPD results of crystalline Form 4 are provided in FIG. 17. A melting temperature at approximately 114° C. was observed using differential scanning calorimetry (FIG. 18). Crystalline Form 4 was moderately hygroscopic and unsolvated.

XRPD measurements of crystalline Form 4 of Compound 1-A were measured. Observed peaks are shown in Table 23. Prominent peaks are listed in Table 24.

TABLE 23
Observed Peaks for Crystalline Form 4

| Diffraction angle | | Intensity |
|---|---|---|
| °2θ (deg) | d space (Å) | (%) |
| 6.2 | 14.3 | 100.0 |
| 8.0 | 11.0 | 15.7 |
| 12.9 | 6.9 | 38.0 |
| 13.2 | 6.7 | 19.8 |
| 14.0 | 6.3 | 39.3 |
| 14.5 | 6.1 | 40.4 |
| 15.6 | 5.7 | 13.6 |
| 16.5 | 5.4 | 67.0 |
| 17.6 | 5.0 | 49.5 |
| 18.0 | 4.9 | 22.9 |
| 18.6 | 4.8 | 66.1 |
| 20.3 | 4.4 | 22.5 |
| 21.2 | 4.2 | 45.9 |
| 22.7 | 3.9 | 80.4 |
| 23.0 | 3.9 | 8.1 |
| 23.3 | 3.8 | 62.1 |
| 24.0 | 3.7 | 23.0 |
| 26.1 | 3.4 | 23.0 |
| 27.0 | 3.3 | 17.1 |
| 27.9 | 3.2 | 8.9 |

TABLE 24
Prominent Peaks for Crystalline Form 4

| Diffraction angle | | Intensity |
|---|---|---|
| °2θ (deg) | d space (Å) | (%) |
| 6.2 | 14.3 | 100.0 |
| 12.9 | 6.9 | 38.0 |
| 14.0 | 6.3 | 39.3 |
| 14.5 | 6.1 | 40.4 |
| 16.5 | 5.4 | 67.0 |
| 17.6 | 5.0 | 49.5 |
| 18.0 | 4.9 | 22.9 |
| 18.6 | 4.8 | 66.1 |
| 20.3 | 4.4 | 22.5 |
| 21.2 | 4.2 | 45.9 |
| 22.7 | 3.9 | 80.4 |
| 23.3 | 3.8 | 62.1 |
| 24.0 | 3.7 | 23.0 |
| 26.1 | 3.4 | 23.0 |

### Example 7: Equilibrium Aqueous Solubility Data for Compound 1 and Compound 1-A

Equilibrium solubility of Compound 1 and 1-A was measured in water at ambient temperature. A known volume of water was placed in a 1-dram glass vial and weight aliquots of Compound 1 or Compound 1-A were added while magnetically stirring at room temperature. If the solid dissolved, another aliquot of solvent was added. The data is summarized below in Table 25.

TABLE 25
Equilibrium Solubility Studies

| | Concentration | Concentration | Concentration |
|---|---|---|---|
| | at 4 hours | at 24 hours | at 48 hours |
| | (mg/mL) | (mg/mL) | (mg/mL) |
| Compound 1 | 0.30 | 0.32 | 0.32 |
| Compound 1-A | 1.45 | 1.43 | 1.42 |

### Example 8: Kinetic Solubility Studies of Compound 1 and Compound 1-A

Kinetic solubility of Compound 1 was measured in both simulated gastric fluid (SGF) at a pH of about 1.2 and in water at 37° C. In SGF, concentrations were below 1 μg/mL up until 6 hours. The resulting solid was form A. In water, a concentration of 0.3 mg/mL was achieved within 2 minutes and concentration after 6 hours was about 0.5 mg/mL.

Similarly, the kinetic solubility of Compound 1-A (Form 1) was measured in both SGF at a pH of about 1.2 and in water at 37° C. In SGF, concentrations were below 1 μg/mL up until 6 hours. The resulting solid was non-crystalline by XRPD. In water, a concentration of 2.3 mg/mL was achieved within 2 minutes and concentration after 6 hours was about 3.1 mg/mL. The resulting solid was found to be crystalline Form 3 of Compound 1-A

TABLE 25
Kinetic Solubility Studies

| | Concentration | Concentration | Concentration | Concentration |
|---|---|---|---|---|
| | Compound | Compound | Compound | Compound |
| | 1 | 1-A | 1 | 1-A |
| Time | in SFG | in SFG | in water | in water |
| (min) | (mg/mL) | (mg/mL) | (mg/mL) | (mg/mL) |
| 2 | 0.00040 | 0.00035 | 0.29 | 2.25 |
| 5 | 0.00018 | 0.00017 | 0.33 | 2.66 |
| 10 | 0.00011 | 0.00011 | 0.30 | 2.98 |
| 30 | 0.00010 | 0.00008 | 0.29 | 3.07 |
| 60 | 0.00007 | 0.00005 | 0.40 | 3.14 |
| 120 | 0.00037 | 0.00028 | 0.41 | 2.28 |
| 240 | 0.00015 | 0.00021 | 0.39 | 2.87 |
| 360 | 0.00010 | 0.00007 | 0.53 | 3.07 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A crystalline form of Compound 1: 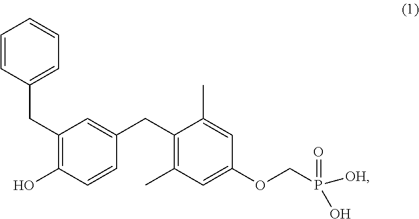 [SMILES: Cc1cc(OCP(=O)(O)O)cc(C)c1Cc1ccc(O)c(Cc2ccccc2)c1]
or a solvate thereof.

2. The crystalline form of claim 1, wherein the crystalline form exhibits an X-ray powder diffraction pattern comprising at least one characteristic peak, wherein said characteristic peak is selected from the group consisting of approximately 5.1, 7.4, 10.1, 12.1, 16.4, 17.3, 18.5, 20.5, 21.4, 21.7, 22.1, 23.8, 24.1, 25.5, and 25.9 degrees 2θ.

3. The crystalline form of claim 2, wherein the crystalline form exhibits an X-ray powder diffraction pattern comprising at least three characteristic peaks, wherein said characteristic peaks are selected from the group consisting of 5.1, 7.4, 10.1, 12.1, 16.4, 17.3, 18.5, 20.5, 21.4, 21.7, 22.1, 23.8, 24.1, 25.5, and 25.9 degrees 2θ.

4. The crystalline form of claim 2 or claim 3, wherein the crystalline form has a melting point of about 161° C.

5. The crystalline form of claim 1, wherein the crystalline form exhibits an X-ray powder diffraction pattern comprising at least one characteristic peak, wherein said characteristic peak is selected from the group consisting of approximately 7.5, 8.1, 10.0, 15.4, 16.7, 18.3, 18.8, 20.2, 21.0, 22.3, 24.1, 25.1, and 26.4 degrees 2θ.

6. The crystalline form of claim 5, wherein the crystalline form exhibits an X-ray powder diffraction pattern comprising at least three characteristic peaks, wherein said characteristic peaks are selected from the group consisting of 7.5, 8.1, 10.0, 15.4, 16.7, 18.3, 18.8, 20.2, 21.0, 22.3, 24.1, 25.1, and 26.4 degrees 2θ.

7. The crystalline form of claim 1, wherein the crystalline form exhibits an X-ray powder diffraction pattern comprising at least one characteristic peak, wherein said characteristic peak is selected from the group consisting of approximately 4.0, 8.0, 11.0, 12.0, 14.4, 14.6, 17.1, 18.5, 19.2, 21.1, 21.7, 24.9, and 25.7 degrees 2θ.

8. The crystalline form of claim 7, wherein the crystalline form exhibits an X-ray powder diffraction pattern comprising at least three characteristic peaks, wherein said characteristic peaks are selected from the group consisting of 4.0, 8.0, 11.0, 12.0, 14.4, 14.6, 17.1, 18.5, 19.2, 21.1, 21.7, 24.9, and 25.7 degrees 2θ.

9. The crystalline form of claim 8 or claim 9, wherein the crystalline form has a DSC endotherm at about 70° C. or 158° C.

10. The crystalline form of claim 1, wherein the crystalline form exhibits an X-ray powder diffraction pattern comprising at least one characteristic peak, wherein said characteristic peak is selected from the group consisting of approximately 76.1, 7.6, 11.3, 12.2, 16.5, 17.0, 17.3, 18.4, 18.6, 19.5, 19.7, 20.7, 21.2, 22.5, 26.0, and 26.3 degrees 2θ.

11. The crystalline form of claim 10, wherein the crystalline form exhibits an X-ray powder diffraction pattern comprising at least three characteristic peaks, wherein said characteristic peaks are selected from the group consisting of 6.1, 7.6, 11.3, 12.2, 16.5, 17.0, 17.3, 18.4, 18.6, 19.5, 19.7, 20.7, 21.2, 22.5, 26.0, and 26.3 degrees 2θ.

12. The crystalline form of claim 10 or claim 11, wherein the crystalline form has a DSC endotherm at about 166° C.

13. The crystalline form of claim 1, wherein the crystalline form exhibits an X-ray powder diffraction pattern comprising at least one characteristic peak, wherein said characteristic peak is selected from the group consisting of approximately 3.3, 5.0, 7.5, 14.9, 16.3, 17.2, 19.4, 21.7, 24.7, 25.5, and 26.6 degrees 2θ.

14. The crystalline form of claim 13, wherein the crystalline form exhibits an X-ray powder diffraction pattern comprising at least three characteristic peaks, wherein said characteristic peaks are selected from the group consisting of 3.3, 5.0, 7.5, 14.9, 16.3, 17.2, 19.4, 21.7, 24.7, 25.5, and 26.6 degrees 2θ.

15. The crystalline form of claim 13 or claim 14, wherein the crystalline form has a DSC endotherm at about 96° C. or 166° C.

16. The crystalline form of claim 1, wherein the crystalline form exhibits an X-ray powder diffraction pattern comprising at least one characteristic peak, wherein said characteristic peak is selected from the group consisting of approximately 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ.

17. The crystalline form of claim 16, wherein the crystalline form exhibits an X-ray powder diffraction pattern comprising at least three characteristic peaks, wherein said characteristic peaks are selected from the group consisting of 8.3, 15.5, 15.6, 15.9, 17.0, 17.3, 17.9, 18.6, 20.8, 22.0, 23.0, 23.6, 23.8, 25.5, 25.7, 26.0, and 26.7 degrees 2θ.

18. The crystalline form of claim 16 or claim 17, wherein the crystalline form has a DSC endotherm at about 96° C.

19. The crystalline form of any one of claims 1 to 18, wherein the crystalline form is unsolvated.

20. The crystalline form of any one of claims 1 to 18, wherein the crystalline form is solvated.

21. A crystalline form of Compound 1-A: 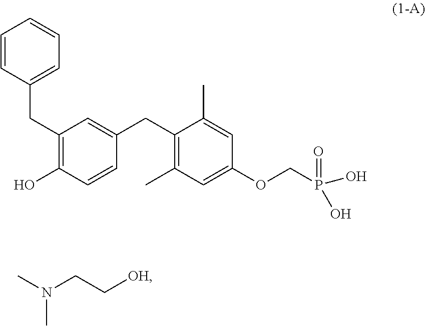
or a solvate thereof.

22. The crystalline form of claim 21, wherein the crystalline form exhibits an X-ray powder diffraction pattern comprising at least one characteristic peak, wherein said characteristic peak is selected from the group consisting of approximately 6.0, 13.3, 14.4, 14.8, 16.1, 17.8, 17.9, 18.7, 20.5, 20.8, 21.7, 22.9, 23.8, 24.9, 25.1, and 26.6 degrees 2θ.

23. The crystalline form of claim 22, wherein the crystalline form exhibits an X-ray powder diffraction pattern comprising at least three characteristic peaks, wherein said characteristic peaks are selected from the group consisting of 6.0, 13.3, 14.4, 14.8, 16.1, 17.8, 17.9, 18.7, 20.5, 20.8, 21.7, 22.9, 23.8, 24.9, 25.1, and 26.6 degrees 2θ.

24. The crystalline form of claim 22 or claim 23, wherein the crystalline form has a melting point of about 156° C.

25. The crystalline form of claim 21, wherein the crystalline form exhibits an X-ray powder diffraction pattern comprising at least one characteristic peak, wherein said characteristic peak is selected from the group consisting of approximately 6.1, 12.2, 13.3, 13.6, 16.5, 17.3, 18.3, 19.9, 20.2, 20.5, 21.4, 21.9, 22.5, 22.8, 23.0, and 25.6 degrees 2θ.

26. The crystalline form of claim 25, wherein the crystalline form exhibits an X-ray powder diffraction pattern comprising at least three characteristic peaks, wherein said characteristic peaks are selected from the group consisting of 6.1, 12.2, 13.3, 13.6, 16.5, 17.3, 18.3, 19.9, 20.2, 20.5, 21.4, 21.9, 22.5, 22.8, 23.0, and 25.6 degrees 2θ.

27. The crystalline form of claim 25 or claim 26, wherein the crystalline form has a melting point of about 139° C.

28. The crystalline form of claim 23, wherein the crystalline form exhibits an X-ray powder diffraction pattern comprising at least one characteristic peak, wherein said characteristic peak is selected from the group consisting of approximately 5.7, 6.2, 15.5, 16.4, 17.0, 18.5, 20.6, 21.2, 22.0, 23.3, and 26.1 degrees 2θ.

29. The crystalline form of claim 28, wherein the crystalline form exhibits an X-ray powder diffraction pattern comprising at least three characteristic peaks, wherein said characteristic peaks are selected from the group consisting of 5.7, 6.2, 15.5, 16.4, 17.0, 18.5, 20.6, 21.2, 22.0, 23.3, and 26.1 degrees 2θ.

30. The crystalline form of claim 28 or claim 29, wherein the crystalline form has a melting point of about 117° C.

31. The crystalline form of claim 21, wherein the crystalline form exhibits an X-ray powder diffraction pattern comprising at least one characteristic peak, wherein said characteristic peak is selected from the group consisting of approximately 6.2, 12.9, 14.0, 14.5, 16.5, 17.6, 18.0, 18.6, 20.3, 21.2, 22.7, 23.3, 24.0, and 26.1 degrees 2θ.

32. The crystalline form of claim 31, wherein the crystalline form exhibits an X-ray powder diffraction pattern comprising at least three characteristic peaks, wherein said characteristic peaks are selected from the group consisting of 6.2, 12.9, 14.0, 14.5, 16.5, 17.6, 18.0, 18.6, 20.3, 21.2, 22.7, 23.3, 24.0, and 26.1 degrees 2θ.

33. The crystalline form of claim 31 or claim 32, wherein the crystalline form has a melting point of about 114° C.

34. The crystalline form of any one of claims 21 to 33, wherein the crystalline form is unsolvated.

35. The crystalline form of any one of claims 21 to 33, wherein the crystalline form is solvated.

36. The crystalline form of claim 35, wherein the crystalline form is a hydrate.

37. The crystalline form of claim 35, wherein the crystalline form is a monohydrate.

38. A pharmaceutical composition comprising a therapeutically effective amount of one or more crystalline forms of any one of claims 1 to 37 and one or more pharmaceutically acceptable excipients.

39. A method of treating a disease or condition in a subject, the method comprising administering to the subject a therapeutically effective amount of a crystalline form of any one of claims 1 to 37, wherein said disease or disorder is selected from the group consisting of steatosis, non-alcoholic fatty liver disease (NAFLD), non-alcoholic steatohepatitis (NASH), liver fibrosis, renal fibrosis, biliary fibrosis, pancreatic fibrosis, chronic kidney disease, diabetic kidney disease, primary sclerosing cholangitis, primary biliary cirrhosis, or idiopathic fibrosis.

40. The method of claim 39, wherein method results in the prevention, treatment, or amelioration, of a fibrosis, fibrotic condition, or fibrotic symptoms.

41. The method of claim 39, wherein method results in the reduction in the amount of extracellular matrix proteins present in one or more tissues of said subject.

42. The method of claim 39, wherein said method results in the reduction in the amount of collagen present in one or more tissues of said subject.

43. The method of claim 39, wherein said administration of said crystalline form results in the reduction in the amount of Type I, Type Ia, or Type III collagen present in one or more tissues of said subject.

44. A method of treating a disease or disorder in a subject, the method comprising administering to the subject a therapeutically effective amount of a solution prepared by dissolving crystalline form of any one of claims 1 to 37 in a pharmaceutically acceptable solvent, wherein said disease or disorder is selected from the group consisting of steatosis, non-alcoholic fatty liver disease (NAFLD), non-alcoholic steatohepatitis (NASH), liver fibrosis, renal fibrosis, biliary fibrosis, pancreatic fibrosis, chronic kidney disease, diabetic kidney disease, primary sclerosing cholangitis, primary biliary cirrhosis, or idiopathic fibrosis.

45. A compound, having the structure 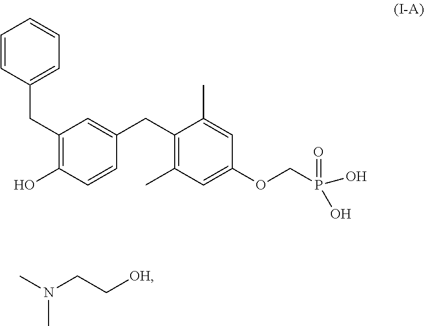
or a solvate thereof.
